(19) United States
(12) Patent Application Publication
(10) Pub. No.: US 2026/0034141 A1
(43) Pub. Date: Feb. 5, 2026

(54) METHODS AND COMPOSITIONS FOR TREATING MALIGNANT CANCERS

(71) Applicant: 6J Biotechnology (Hong Kong) Limited, Hong Kong (HK)

(72) Inventors: Jianbo YUE, Hong Kong (HK); Peng WANG, Hong Kong (HK); Qingru ZHANG, Hong Kong (HK)

(21) Appl. No.: 18/793,982

(22) Filed: Aug. 5, 2024

(51) Int. Cl. A61K 31/541; A61K 31/53; A61K 31/5377; A61K 45/06; A61P 35/00

(52) U.S. Cl. CPC A61K 31/541; A61K 31/53; A61K 31/5377; A61K 45/06; A61P 35/00

(57) ABSTRACT

A series of 1,3,5-triazine compounds that exhibit potent inhibition of endosomal trafficking and autophagy is provided. These compounds effectively suppress cancer cell proliferation, growth, and migration, induce cell cycle arrest at the G0/G1 phase, promote cancer cell death via non-apoptotic pathways, and inhibit tumor sphere formation. Moreover, select compounds within this series demonstrate significant inhibition of tumor growth and metastasis in mouse models of lung cancer and melanoma. Additionally, they modulate macrophage polarization and the tumor microenvironment by regulating cytokine secretion. These findings highlight the potential of 1,3,5-triazine compounds as promising antitumor agents.

### FIELD OF THE INVENTION

The present invention generally relates to at least the fields of molecular biology, cell biology, medicine, life sciences research, cancer research, drug models, therapeutic testing, and so forth.

### BACKGROUND OF THE INVENTION

Malignant cancers are a significant cause of morbidity and mortality worldwide, posing a substantial challenge to healthcare systems. Traditional cancer therapies, such as chemotherapy and radiation, often result in severe side effects and are not always effective, especially in advanced stages of the disease. Therefore, there is an ongoing need for the development of novel therapeutic agents with improved efficacy and reduced toxicity.

Recent advances in cancer biology have highlighted the role of intracellular trafficking processes, such as endosomal trafficking and autophagy, in cancer cell survival, proliferation, and metastasis. Dysregulation of these processes is frequently observed in cancer cells, contributing to their uncontrolled growth and resistance to conventional therapies. Consequently, targeting these pathways presents a promising strategy for cancer treatment.

Despite these advances, several critical issues remain unresolved in the field. Current cancer treatments often fail to address the complexities of tumor microenvironments, leading to incomplete eradication of cancer cells and eventual relapse. Additionally, many therapeutic agents exhibit significant toxicity, limiting their clinical application. Resistance to apoptosis-inducing drugs is another major challenge, as cancer cells frequently develop mechanisms to evade cell death.

### SUMMARY OF THE INVENTION

It is an objective of the present invention to provide more potential endosomal trafficking inhibitors to solve the aforementioned technical problems.

In a first aspect, the present invention provides a series of novel 1,3,5-triazine derivatives, including the following structural formulas:

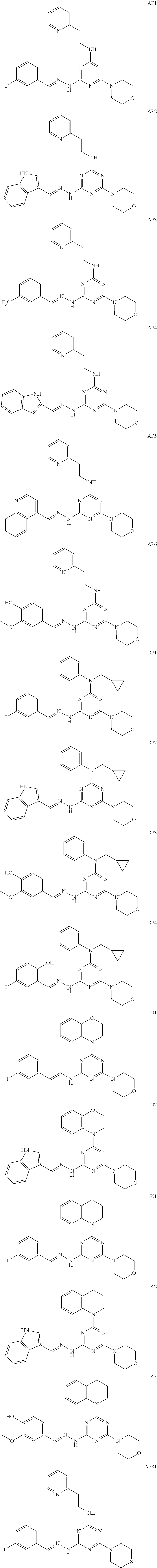

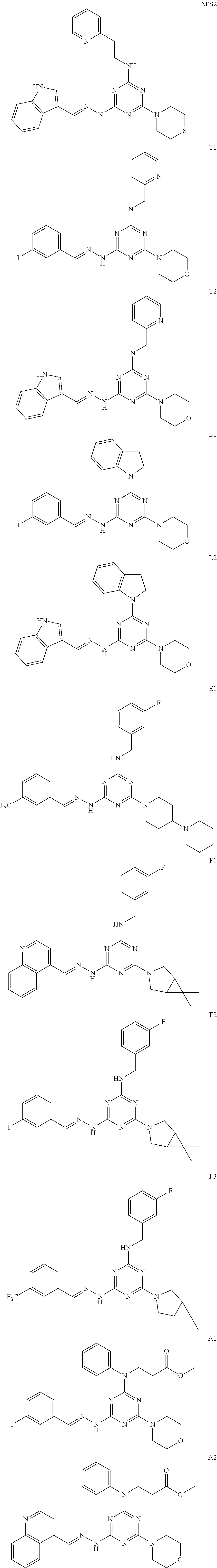

In one embodiment, the 1,3,5-triazine derivative induces macrophages to polarize into M1 macrophages.

In one embodiment, the 1,3,5-triazine derivative can treat cancer cells through the following mechanisms: inhibits endosomal trafficking of the malignant cancer cells, resulting in induction of necrosis in the malignant cancer cells; inhibits autophagy in the malignant cancer cells; inhibits proliferation of the malignant cancer cells; inhibits colony formation, migration, and tumor sphere formation in the malignant cancer cells; inhibits metastasis of the malignant cancer cells.

In a second aspect, the present invention relates to a method of treating a malignant cancer in a subject in need thereof, in particular but not exclusively includes administering a therapeutically effective amount of a pharmaceutical composition to the subject. The pharmaceutical composition includes a 1,3,5-triazine derivative and a pharmaceutically acceptable carrier.

In one embodiment, the malignant cancers refer to any cancer that is invasive and can spread to other parts of the body. Common types of malignant cancer include, but not limit to carcinoma, sarcoma, leukemia, lymphoma and myeloma. For instance, the malignant cancer can be a breast cancer, lung cancer, colorectal cancer, prostate cancer, pancreatic cancer, liver cancer, esophageal cancer, gastric cancer, ovarian cancer, cervical cancer, endometrial cancer, renal cancer, bladder cancer, thyroid cancer, melanoma, non-Hodgkin lymphoma, Hodgkin lymphoma, multiple myeloma, leukemia, glioblastoma, astrocytoma, medulloblastoma, meningioma, sarcoma, bone cancer, head and neck cancer, testicular cancer, oral cancer, anal cancer, mesothelioma, neuroblastoma, retinoblastoma, and cholangiocarcinoma.

Preferably, the malignant cancer is selected from melanoma or lung carcinoma.

In another embodiment, the cancer comprises lung cancers. Lung cancer includes non-small cell lung cancer (e.g., adenocarcinoma, squamous cell carcinoma, large cell carcinoma) and small cell lung cancer.

In one embodiment, the 1,3,5-triazine derivative includes the following structural formulas:

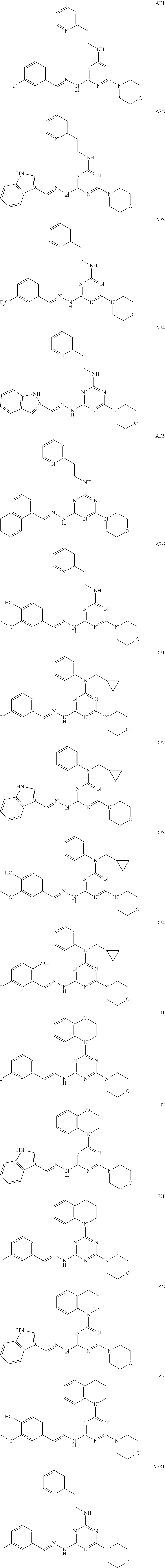

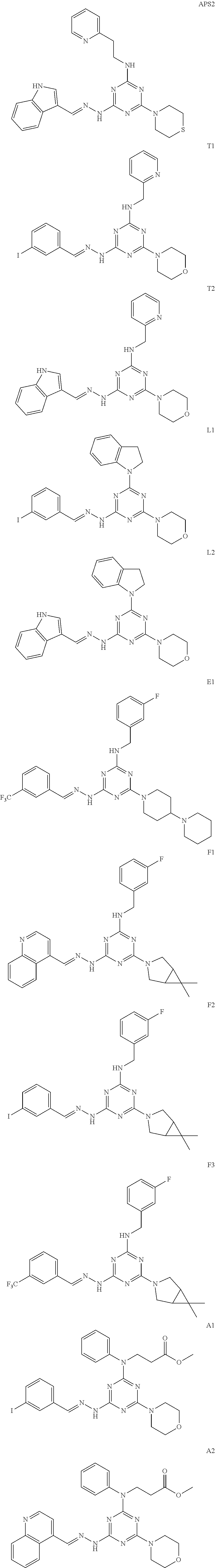

In one embodiment, the 1,3,5-triazine derivative induces macrophages to polarize into MI macrophages.

In one embodiment, the pharmaceutically acceptable carrier is selected from the group consisting of diluents, excipients, and stabilizers.

In one embodiment, the pharmaceutical composition inhibits endosomal trafficking of the malignant cancer cells, resulting in induction of necrosis in the malignant cancer cells.

In one embodiment, the pharmaceutical composition inhibits autophagy in the malignant cancer cells.

In one embodiment, the pharmaceutical composition inhibits proliferation of the malignant cancer cells.

In one embodiment, the pharmaceutical composition inhibits colony formation, migration, and tumor sphere formation in the malignant cancer cells.

In one embodiment, the pharmaceutical composition inhibits metastasis of the malignant cancer cells.

In another embodiment, the pharmaceutical composition further includes a therapeutic agent.

In one embodiment, the therapeutic agent includes an anti-PD1 antibody, a chemotherapeutic agent, or an immunotherapeutic agent.

In one embodiment, the ratio of the 1,3,5-triazine derivative to the therapeutic agent is 250:1.

In one embodiment, the subject is a mammal.

In one embodiment, the therapeutically effective amount of the 1,3,5-triazine derivative is between 30-50 mg/kg per day.

In one embodiment, the 1,3,5-triazine derivative is present in an amount of 30-50 mg per dosage unit.

In one embodiment, the pharmaceutical composition is administered once a day.

In one embodiment, the pharmaceutical composition is administered via oral, intravenous, or intramuscular routes.

In one embodiment, the pharmaceutical composition is in the form of powder, a tablet, a capsule, or an injectable solution.

In a third aspect, the present invention provides a pharmaceutical composition including a 1,3,5-triazine derivative in an amount effective for treating a malignant cancer in a subject in need thereof, and a pharmaceutically acceptable addition.

### BRIEF DESCRIPTION OF THE DRAWINGS

Embodiments of the invention are described in more details hereinafter with reference to the drawings, in which:

FIG. 1A shows a synthetic route of 1,3,5-triazine derivatives. FIG. 1B shows R₂ group of the synthetic 1,3,5-triazine derivatives. Substitutions on 2-, 4-, and 6-position were performed to obtain more compounds;

FIG. 2 shows images of cells treated with various 1,3,5-triazine derivatives of the present invention observed under 20× objective lens, highlighting the presence of vacuoles. The scale bar is 100 μm;

FIG. 3A depicts the Western blot results indicating the levels of LC3 and P62 in HeLa cells treated with V1, Vac, or other synthetic 1,3,5-triazine derivatives. FIG. 3B depicts the Western blot results indicating the levels of LC3 and P62 in HeLa cells treated with V1, AP1, or AP2 at indicated concentrations;

FIG. 4A shows bright-field images of Hela cells, displaying vacuoles following treatment with V1, API or AP2 (at specified concentrations) for 24 h. The scale bar is 1 μm. FIG. 4B shows results and quantification of DQ-BSA trafficking assay conducted on HeLa cells treated with 100 nM Baf A1, or with V1, API or AP2 (all at 1 μM) for 4 h;

FIG. 5A shows fluorescence staining images captured with a Zeiss 880 microscope at 63× objective. The scale bar is 5 μm. FIG. 5B depicts quantification of the LC3 puncta in FIG. 5A;

FIG. 6A depicts results of MTT assay conducted on HeLa, A375, A549 or LLC cells treated with different concentration of 1,3,5-triazine derivatives of the present invention for 48 hr. FIG. 6B depicts cell proliferation index of A375, A549 and Hela cells treated with different concentration of 1,3,5-triazine derivatives of the present invention for 24 and 48 hr. FIG. 6C depicts results and quantification of PI co-staining conducted on A375, A549 and Hela cells treated with different concentration of 1,3,5-triazine derivatives of the present invention for 24 and 48 hr;

FIG. 7 shows colony formation of A549 cells treated with API or AP2 and incubated for 7 days;

FIG. 8A shows images from the transwell migration assay and quantification of migrated cells. The scale bar is 200 μm. FIG. 8B shows images from the wound healing assay and quantification of closure area by Image J. ((*p<0.05, **p<0.01, ***p<0.001. ns: no significance);

FIG. 9A the flow cytometry results of A549 cells treated with 0, 0.1, 0.5, or 1 μM of API or AP2 for 48 hr. FIG. 9B depicts qualification of the percentage of each phase (G₁, S and G₂);

FIG. 10A shows fluorescence staining images showing Annexin-V⁺PI⁺ cell treated with API or AP2. FIG. 10B shows the Western blot results indicating the levels of PARP and cl-PARP in cancer cells treated with API or Phloretin. FIG. 10C shows appearance of tumor spheres after treatment with 20 μM of AP1 or AP2, and Hoechst and PI stainings were performed at the end point. Images are acquired with a Nikon Ti-s microscope using a 20× objective lens. The scale bar is 100 μm. The quantification of total PI and Hoechst fluorescence was performed by image J. (***p<0.001);

FIG. 11A shows tumors images from xenograft nude mice (with A549 cells) treated with or without AP1 at the endpoint. FIG. 11B shows changes in tumor volume over 21 days. FIG. 11C shows tumor weight of the nude mice treated with or without AP1. FIG. 11D shows changes in body weight over 21 days. The tumor volume and body weight were measured twice per week (*p<0.05, **p<0.01, ***p<0.001. ns: no significance);

FIG. 12A shows tumors images from xenograft C57/BL6 mice (with LLC cells) treated with or without API at the endpoint. FIG. 12B shows changes in tumor volume over 18 days. FIG. 12C shows tumor weight of the C57/BL6 mice treated with or without AP1. FIG. 12D shows changes in body weight over 18 days. The tumor volume and body weight were measured twice a week (*p<0.05, **p<0.01, ***p<0.001). FIG. 12E depicts expression levels of various genes in primary tumors analyzed by qRT-PCR;

FIG. 13A shows tumors images from xenograft C57/BL6 mice (with B16F10 cells) treated with or without AP1 at the endpoint. FIG. 13B shows changes in tumor volume over 16 days. FIG. 13C shows tumor weight of the C57/BL6 mice treated with or without AP1. FIG. 13D shows changes in body weight over 16 days. FIG. 13E depicts expression levels of various genes in primary tumors analyzed by qRT-PCR;

FIG. 14A shows tumors images from LLC xenografted C57/BL6 mice treated with AP1, IgG, or PD-1, or their combination thereof. FIG. 14B shows changes in tumor volume over 17 days. FIG. 14C shows tumor weight of the C57/BL6 mice treated with AP1, IgG, or PD-1, or their combination thereof. FIG. 14D shows changes in body weight over 17 days. The tumor volume and body weight were measured twice a week. (*p<0.05, **p<0.01, ***p<0.001. ns: no significance); and

FIG. 15A presents images of mice, illustrating luminescent signals in each group at 4-day intervals. FIG. 15B shows lung image showing the luminescent signals treated with or without AP1 treatment. FIG. 15C shows changes in body weight over 19 days.

### DETAILED DESCRIPTION

In the following description, methods, compositions, and/or usages are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In a first aspect, the present invention provides a method for treating malignant cancer in a subject in need thereof by administering a therapeutically effective amount of a pharmaceutical composition. This pharmaceutical composition includes a 1,3,5-triazine derivative and a pharmaceutically acceptable carrier. The method is specifically designed to address a wide range of malignant cancers, including but not limited to breast cancer, lung cancer, colorectal cancer, prostate cancer, pancreatic cancer, liver cancer, esophageal cancer, gastric cancer, ovarian cancer, cervical cancer, endometrial cancer, renal cancer, bladder cancer, thyroid cancer, melanoma, non-Hodgkin lymphoma, Hodgkin lymphoma, multiple myeloma, leukemia, glioblastoma, astrocytoma, medulloblastoma, meningioma, sarcoma, bone cancer, head and neck cancer, testicular cancer, oral cancer, anal cancer, mesothelioma, neuroblastoma, retinoblastoma, and cholangiocarcinoma. In particular, the method has shown efficacy in treating melanoma and lung carcinoma.

The 1,3,5-triazine derivative employed in this method includes the following structural formulas:

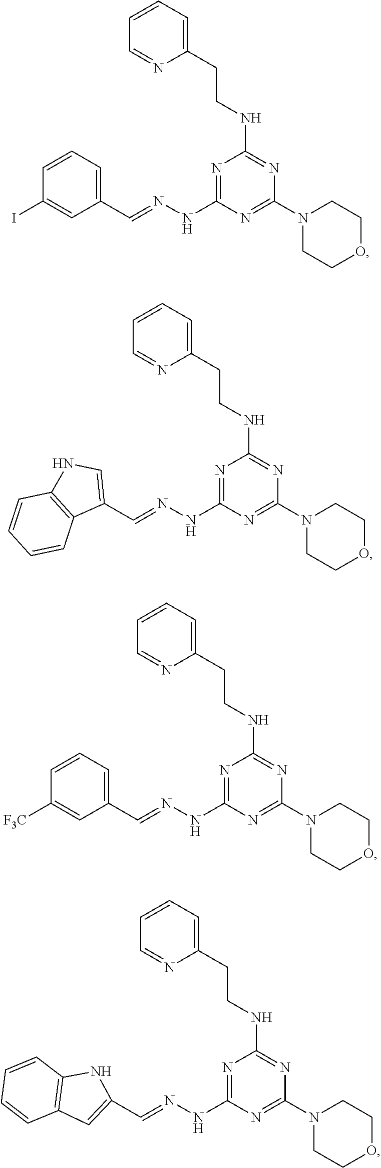 [SMILES: Ic1cccc(/C=N/Nc2nc(NCCc3ccccn3)nc(N3CCOCC3)n2)c1 ; C(=N\Nc1nc(NCCc2ccccn2)nc(N2CCOCC2)n1)/c1c[nH]c2ccccc12 ; FC(F)(F)c1cccc(/C=N/Nc2nc(NCCc3ccccn3)nc(N3CCOCC3)n2)c1 ; C(=N\Nc1nc(NCCc2ccccn2)nc(N2CCOCC2)n1)/c1cc2ccccc2[nH]1]

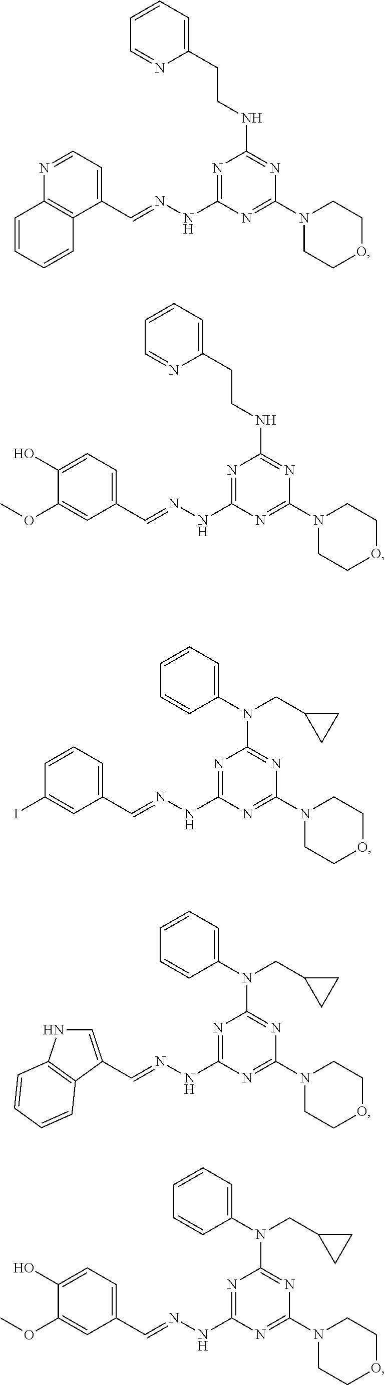 [SMILES: C(=N\Nc1nc(NCCc2ccccn2)nc(N2CCOCC2)n1)/c1ccnc2ccccc12 ; COc1cc(/C=N/Nc2nc(NCCc3ccccn3)nc(N3CCOCC3)n2)ccc1O ; Ic1cccc(/C=N/Nc2nc(N3CCOCC3)nc(N(CC3CC3)c3ccccc3)n2)c1 ; C(=N\Nc1nc(N2CCOCC2)nc(N(CC2CC2)c2ccccc2)n1)/c1c[nH]c2ccccc12 ; COc1cc(/C=N/Nc2nc(N3CCOCC3)nc(N(CC3CC3)c3ccccc3)n2)ccc1O]

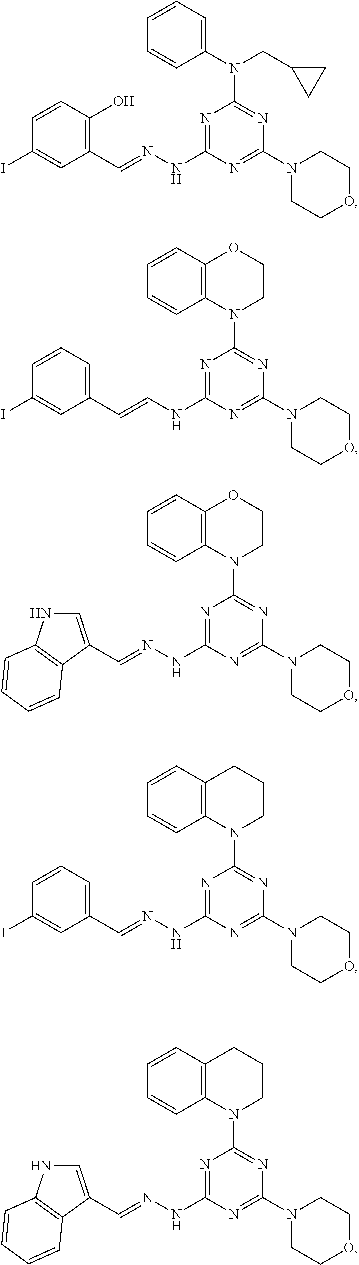 [SMILES: Oc1ccc(I)cc1/C=N/Nc1nc(N2CCOCC2)nc(N(CC2CC2)c2ccccc2)n1 ; Ic1cccc(/C=C/Nc2nc(N3CCOCC3)nc(N3CCOc4ccccc43)n2)c1 ; C(=N\Nc1nc(N2CCOCC2)nc(N2CCOc3ccccc32)n1)/c1c[nH]c2ccccc12 ; Ic1cccc(/C=N/Nc2nc(N3CCOCC3)nc(N3CCCc4ccccc43)n2)c1 ; C(=N\Nc1nc(N2CCOCC2)nc(N2CCCc3ccccc32)n1)/c1c[nH]c2ccccc12]

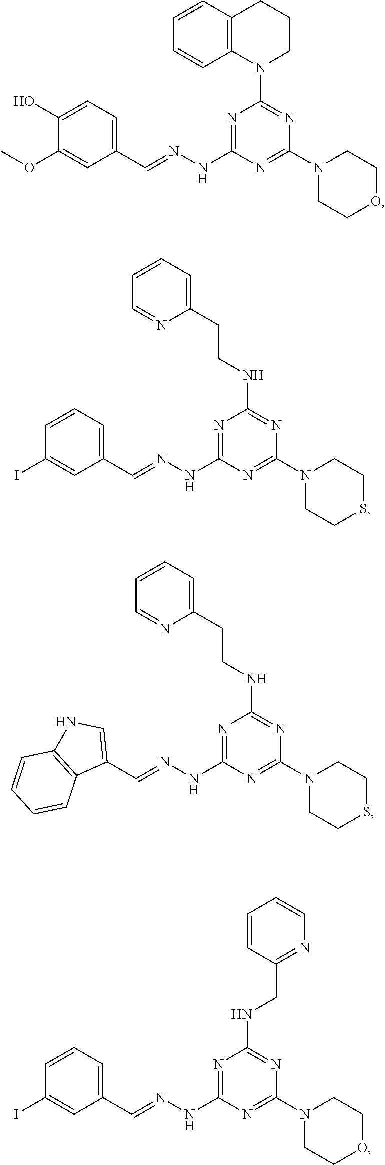 [SMILES: COc1cc(/C=N/Nc2nc(N3CCOCC3)nc(N3CCCc4ccccc43)n2)ccc1O ; Ic1cccc(/C=N/Nc2nc(NCCc3ccccn3)nc(N3CCSCC3)n2)c1 ; C(=N\Nc1nc(NCCc2ccccn2)nc(N2CCSCC2)n1)/c1c[nH]c2ccccc12 ; Ic1cccc(/C=N/Nc2nc(NCc3ccccn3)nc(N3CCOCC3)n2)c1]

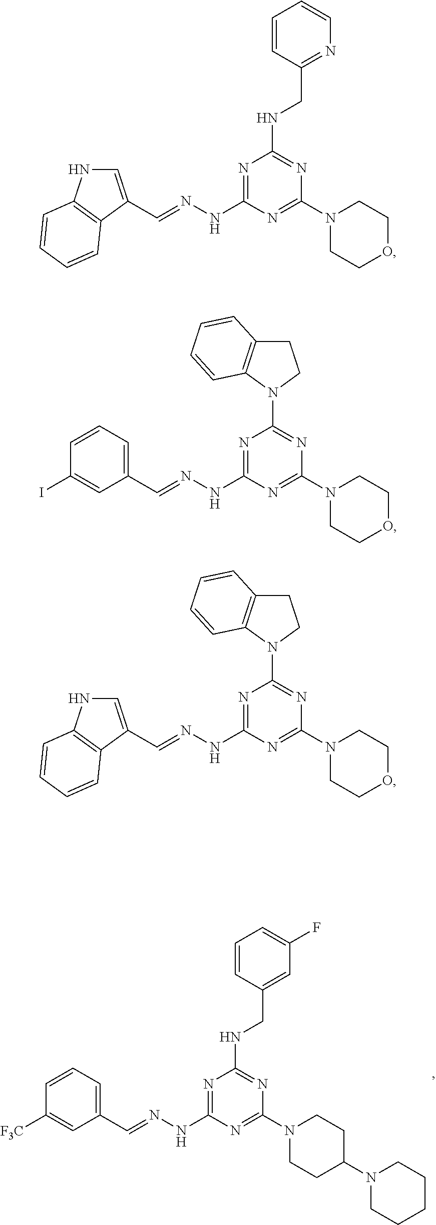 [SMILES: C(=N\Nc1nc(NCc2ccccn2)nc(N2CCOCC2)n1)/c1c[nH]c2ccccc12 ; Ic1cccc(/C=N/Nc2nc(N3CCOCC3)nc(N3CCc4ccccc43)n2)c1 ; C(=N\Nc1nc(N2CCOCC2)nc(N2CCc3ccccc32)n1)/c1c[nH]c2ccccc12 ; Fc1cccc(CNc2nc(N/N=C/c3cccc(C(F)(F)F)c3)nc(N3CCC(N4CCCCC4)CC3)n2)c1]

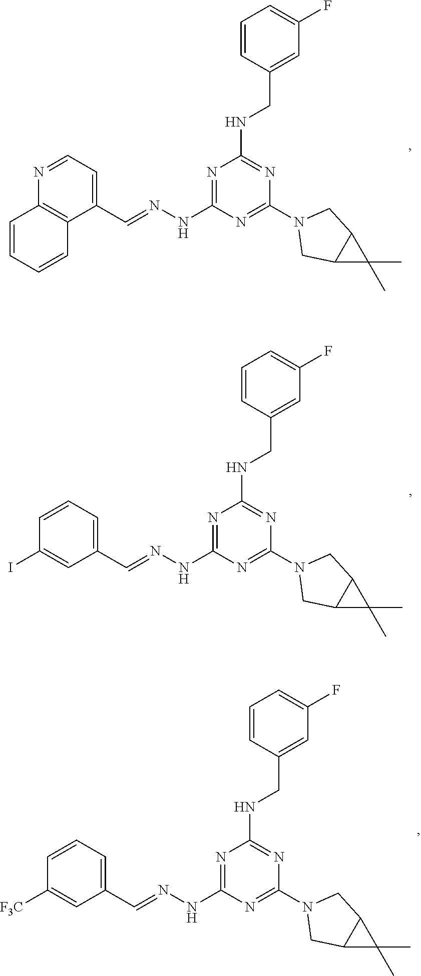 [SMILES: CC1(C)C2CN(c3nc(NCc4cccc(F)c4)nc(N/N=C/c4ccnc5ccccc45)n3)CC21 ; CC1(C)C2CN(c3nc(NCc4cccc(F)c4)nc(N/N=C/c4cccc(I)c4)n3)CC21 ; CC1(C)C2CN(c3nc(NCc4cccc(F)c4)nc(N/N=C/c4cccc(C(F)(F)F)c4)n3)CC21]

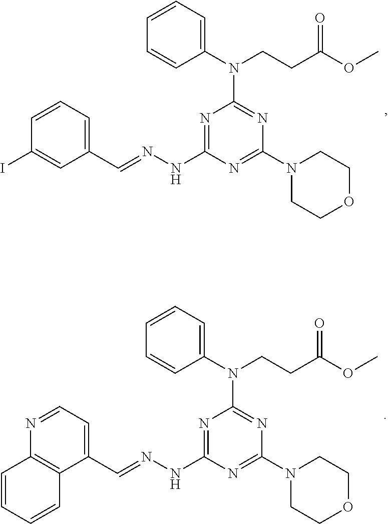 [SMILES: COC(=O)CCN(c1ccccc1)c1nc(N/N=C/c2cccc(I)c2)nc(N2CCOCC2)n1 ; COC(=O)CCN(c1ccccc1)c1nc(N/N=C/c2ccnc3ccccc23)nc(N2CCOCC2)n1]

This derivative plays a significant role in inducing macrophages to polarize into M1 macrophages, which are known for their anti-tumor properties. The pharmaceutical composition includes a pharmaceutically acceptable carrier that can be selected from a group consisting of diluents, excipients, and stabilizers, ensuring the stability and effectiveness of the therapeutic formulation.

One of the key mechanisms of action for the pharmaceutical composition is its ability to inhibit endosomal trafficking of malignant cancer cells, leading to the induction of necrosis in these cells. Additionally, the composition inhibits autophagy and proliferation of the malignant cancer cells, further contributing to its therapeutic effects. The pharmaceutical composition is also effective in inhibiting colony formation, migration, and tumor sphere formation in malignant cancer cells, thereby reducing their ability to metastasize.

Furthermore, the method described includes the potential for the pharmaceutical composition to be combined with other therapeutic agents, enhancing its efficacy. These therapeutic agents can include anti-PD1 antibodies, chemotherapeutic agents, or immunotherapeutic agents, providing a multi-faceted approach to cancer treatment.

In summary, this invention introduces a comprehensive method for treating malignant cancers using a 1,3,5-triazine derivative-based pharmaceutical composition. This composition not only targets a broad spectrum of cancers but also employs multiple mechanisms to inhibit cancer cell survival, proliferation, and metastasis. The method's adaptability to include additional therapeutic agents further enhances its potential efficacy, offering a promising avenue for cancer treatment.

In accordance with a second aspect of the present invention, a pharmaceutical composition including a 1,3,5-triazine derivative in an amount effective along with a pharmaceutically acceptable addition for treating a malignant cancer in a subject in need thereof is introduced. The 1,3,5-triazine derivative includes the aforementioned structural formulas. This composition is designed to target and treat a variety of malignant cancers, including but not limited to breast cancer, lung cancer, colorectal cancer, prostate cancer, pancreatic cancer, liver cancer, esophageal cancer, gastric cancer, ovarian cancer, cervical cancer, endometrial cancer, renal cancer, bladder cancer, thyroid cancer, melanoma, non-Hodgkin lymphoma, Hodgkin lymphoma, multiple myeloma, leukemia, glioblastoma, astrocytoma, medulloblastoma, meningioma, sarcoma, bone cancer, head and neck cancer, testicular cancer, oral cancer, anal cancer, mesothelioma, neuroblastoma, retinoblastoma, and cholangiocarcinoma.

The pharmaceutical composition is formulated to include a pharmaceutically acceptable carrier, which may be selected from a group consisting of diluents, excipients, and stabilizers. These carriers ensure the stability, bioavailability, and effectiveness of the 1,3,5-triazine derivative in treating malignant cancers. The administration of the pharmaceutical composition can be conducted via various routes, including oral, intravenous, or intramuscular routes, depending on the specific needs and condition of the patient.

This approach leverages the therapeutic potential of the 1,3,5-triazine derivative, providing a versatile and effective treatment option for a broad spectrum of malignant cancers. The inclusion of pharmaceutically acceptable carriers enhances the composition's usability and efficacy, ensuring that it can be administered in a manner that maximizes patient comfort and treatment outcomes.

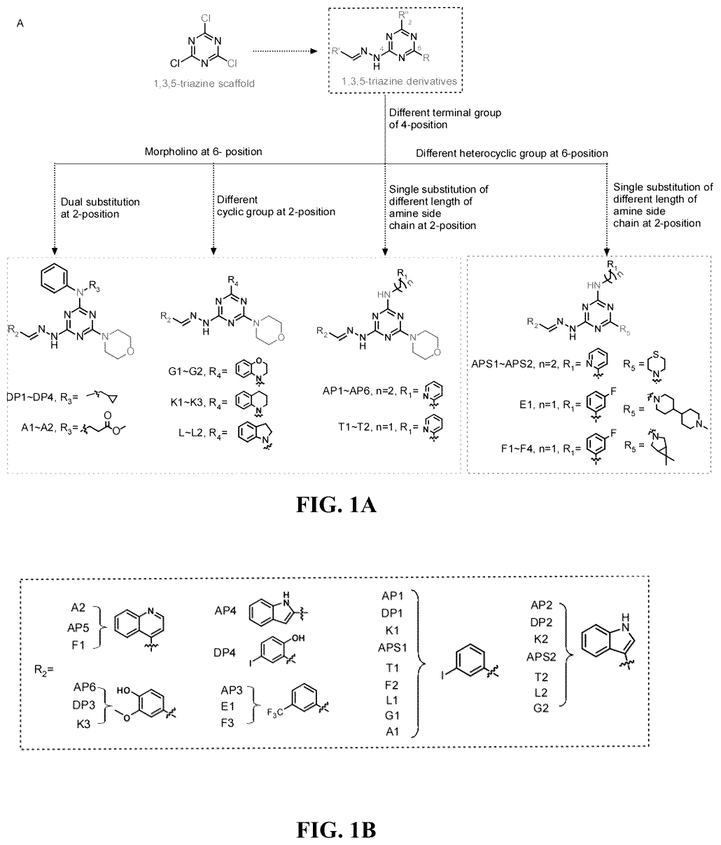

The 1,3,5-triazine derivative includes a series of novel 1,3,5-triazine derivatives. FIGS. 1A-1B illustrates the design strategies and structures of these derivatives, with diverse groups introduced at the 2-, 4-, and 6-positions.

Typically, in all compounds, a hydrazone group with various heterocyclic groups or a substituted benzene ring is attached at the terminal of the 4-position. At the 6-position, morpholine group is introduced, followed by sidechains of varying lengths (n=1 or n=2) with diverse substituted benzene rings or heterocyclic groups at the 2-position terminus. Alternatively, different heterocyclic groups or a substituted benzene ring are directly introduced at the 2-position, or a dual substitution is implemented with a benzene ring and either a cyclopropylethyl or methyl butyrate group at the 2-position.

Furthermore, thiomorpholino, 1-Methyl-4,4′-bipiperidine, or 6,6-Dimethyl-3-azabicyclo[3.1.0]hexane is introduced at the 6-position, followed by sidechains of varying lengths (n=1 or n=2) featuring a 3-fluorobenzene ring or a 2-pyridine group at the 2-position terminus.

The synthetic pathway and reaction conditions for 1,3,5-triazine compounds are as follows:

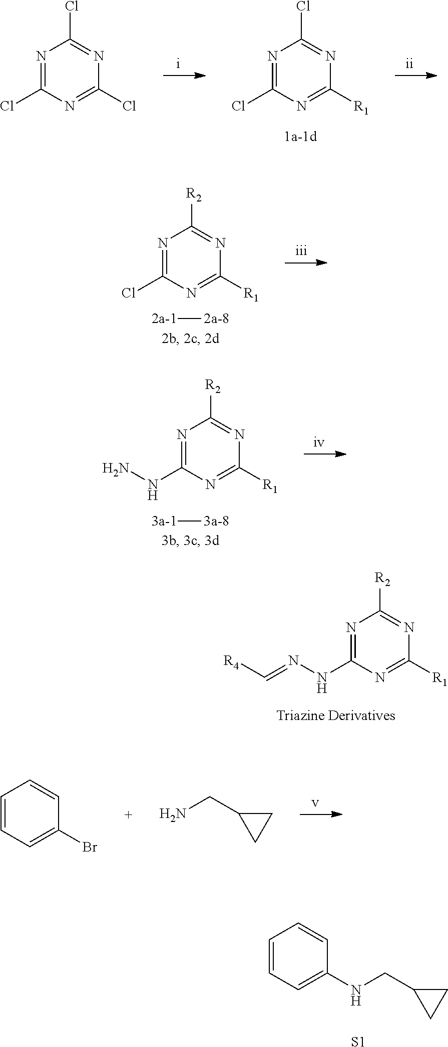

i: addition of anime, K₂CO₃, acetone at room temperature;
ii: addition of anime, K₂CO₃, acetone at room temperature;
iii: addition of NH₂NH₂, tetrahydrofuran reflux;
iv: addition of aldehyde, EtOH, EtCOOH at room temperature;
v: Copper (I) iodide (Cul), anhydrous DMF, CeCO₃ at 90° C.

Compounds 1a-1d are prepared by slowly adding morpholine, thiomorpholine, 4-piperidinopiperidine, or 6,6-dimethyl-3-azabicyclo[3.1.0]hexane (1 equivalent) in acetone solution dropwise to a mixture of cyanuric chloride (1.1 equivalents) and K₂CO₃ (1.5 equivalents) in acetone solution on an ice bath. The resulting mixture is stirred overnight at room temperature. The solvent is removed under vacuum, and the resulting solid is dissolved in ethyl acetate (EA), then washed three times with water and dried over Na₂SO₄. The crude product is subsequently purified by column chromatography, yielding compounds 1a, 1b, 1c, or 1d as white solids in yields of 75-85%, respectively.

Compound S1 is prepared by dissolving bromobenzene (1 eq), CeSO₃, and CuI (0.1 eq) in anhydrous N,N-Dimethylformamide (DMF), then cyclopropylmethanamine (2 eq) in anhydrous DMF is added dropwise into the mixture, which was then stirred overnight at 100° C. After cooling to room temperature, water is added, and the product is extracted with ethyl acetate (EA), washed three times with water, and dried over Na₂SO₄. The crude product is further purified by column chromatography to yield compound S1 as a colorless oil in 80% yield.

Compounds 2a-1 to 2a-8, 2b, 2c and 2d are prepared by adding (1) 3-aminobenzotrifluoride, aniline, 3-fluoroaniline, 2-pyridin-2-ylethanamine, 2-picolylamine, benzomorpholine 1,2,3,4-tetrahydroquinoline, indoline, compound S1 or methyl 3-aminopropanoate; or (2) 2-pyridin-2-ylethanamine; or (3) 3-fluoroaniline; or (4) 3-fluoroaniline in acetone solution (1 eq) dropwise into compounds 1a, 1b, 1c, or 1d (1.1 eq) and K₂CO₃ (1.5 eq) in acetone solution on an ice bath, respectively. The mixture is stirred overnight at room temperature. The solvent is evaporated under vacuum, and the resulting solid is dissolved in ethyl acetate (EA), then washed three times with water and dried over Na₂SO₄. The crude product is further purified by column chromatography to yield compounds 2a-1 to 2a-8, 2b, 2c, or 2d as white solids, with yields ranging from 60% to 80%, respectively.

Compounds 3a-1 to 3a-8, 3b, 3c and 3d are prepared by adding NH₂NH₂ (10 eq) in THE solution dropwise into each THF solution with compounds 2a-1˜2a-8, 2b, 2c or 2d (1 eq), the mixture is stirred and reflux for 1-2 hours. The solvent is evaporated under vacuum, and the resulting solid is dissolved in ethyl acetate (EA), then washed three times with water and dried over Na₂SO₄. The crude product is concentrated and used for the next step without further purification.

Afterward, the aldehyde (1.1 equivalents) in ethanol solution is added dropwise to each solution of compounds 3a-1 to 3a-10, 3b, 3c, or 3d (1 equivalent) in ethanol with stirring. Then, 5-6 drops of acetic acid are added. The mixture is stirred at room temperature for 1-2 hours. Subsequently, the solvent is evaporated under vacuum, and the crude product is further purified by column chromatography to yield a white to light yellow solid, named compounds AP1-AP6, DP1-DP4, G1-G2, K1-K3, APS1-APS2, T1-T2, L1-L2, E1, F1-F3, A1-A2. Each is described as follows:

### (E)-4-(2-(3-iodobenzylidene) hydrazineyl)-6-morpholino-N-(2-(pyridin-2-yl)ethyl)-1,3,5-triazin-2-amine (AP1)

Compound AP1 is synthesized by compound 3a-4 and 3-iodobenzaldehyde, resulting in a white solid with a 75% yield, and it has the following structure:

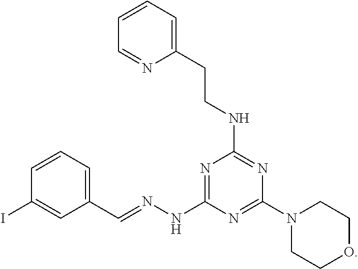 [SMILES: Ic1cccc(/C=N/Nc2nc(NCCc3ccccn3)nc(N3CCOCC3)n2)c1]

Its ¹H NMR (300 MHz, DMSO-d6) exhibits peaks at δ 10.84 (d, J=50.9 Hz, 1H), 8.50 (d, J=5.4 Hz, 1H), 8.07-7.95 (m, 2H), 7.78-7.54 (m, 3H), 7.35-6.96 (m, 4H), 3.81-3.51 (m, 10H), 3.02-2.93 (m, 2H).

The ¹³C NMR (151 MHz, DMSO-d6) shows signals at δ 166.27, 165.22, 164.45, 159.99, 149.52, 140.22, 137.93, 137.70, 136.87, 131.32, 123.63, 121.86, 95.67, 66.50, 43.68, 40.67, 37.94.

HRMS (ESI) gives m/z [M+H]⁺ calculated for C₂₁H₂₃IN₈O: 531.1040, found: 530.1013.

### (E)-4-(2-((1H-indol-3-yl)methylene) hydrazineyl)-6-morpholino-N-(2-(pyridin-2-yl)ethyl)-1,3,5-triazin-2-amine (AP2)

Compound AP2 is synthesized by compound 3a-4 and indole-3-carboxaldehyde, resulting in a white solid with a 75% yield, and it has the following structure:

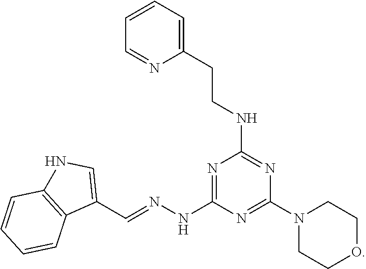 [SMILES: C(=N\Nc1nc(NCCc2ccccn2)nc(N2CCOCC2)n1)/c1c[nH]c2ccccc12]

Its ¹H NMR (400 MHz, Chloroform-d) exhibits peaks at δ 8.50 (s, 2H), 7.98 (s, 2H), 7.55 (s, 1H), 7.39-7.33 (m, 1H), 7.29 (s, 1H), 7.23-7.02 (m, 4H), 3.82 (s, 6H), 3.71 (t, J=4.7 Hz, 4H), 3.07 (s, 2H).

The ¹³C NMR (126 MHz, Chloroform-d) shows signals at δ 159.50, 149.28, 138.98, 136.54, 123.53, 123.14, 121.49, 121.01, 111.26, 66.88, 43.68, 40.35, 37.79.

HRMS (ESI) gives m/z [M+H]⁺ calculated for C₂₃H₂₅N₉O: 444.2235, found: 444.2255.

### (E)-4-morpholino-N-(2-(pyridin-2-yl)ethyl)-6-(2-(3-(trifluoromethyl)benzylidene) hydrazineyl)-1,3,5-triazin-2-amine (AP3)

Compound AP3 is synthesized by compound 3a-4 and 3-(trifluoromethyl)benzaldehyde, resulting in a white solid with a 75% yield, and it has the following structure:

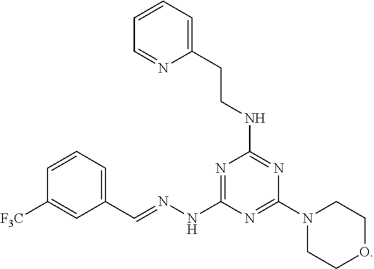 [SMILES: FC(F)(F)c1cccc(/C=N/Nc2nc(NCCc3ccccn3)nc(N3CCOCC3)n2)c1]

Its ¹H NMR (300 MHz, DMSO-d6) exhibits peaks at δ 10.95 (d, J=41.2 Hz, 1H), 8.50 (d, J=4.9 Hz, 1H), 8.16 (d, J=10.6 Hz, 1H), 7.92 (d, J=20.2 Hz, 2H), 7.78-7.59 (m, 3H), 7.38-7.03 (m, 3H), 3.81-3.50 (m, 10H), 3.00 (q, J=7.5 Hz, 2H).

The ¹³C NMR (151 MHz, DMSO-d6) shows signals at δ 166.27, 165.24, 164.49, 159.99, 149.51, 140.24, 136.86, 130.72, 130.34, 130.09, 129.88, 125.57, 123.62, 122.66, 121.87, 66.50, 43.69, 37.94.

HRMS (ESI) gives m/z [M+H]⁺ calculated for C₂₂H₂₃F₃N₈O: 473.1947, found: 473.1927.

### (E)-4-(2-((1H-indol-2-yl)methylene) hydrazineyl)-6-morpholino-N-(2-(pyridin-2-yl)ethyl)-1,3,5-triazin-2-amine (AP4)

Compound AP4 is synthesized by compound 3a-4 and 1H-indole-2-carbaldehyde, resulting in a white solid with a 68% yield, and it has the following structure:

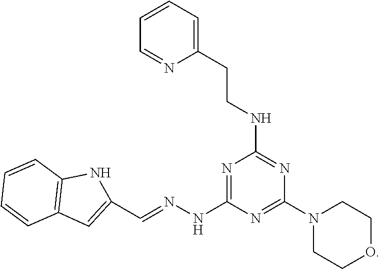 [SMILES: C(=N\Nc1nc(NCCc2ccccn2)nc(N2CCOCC2)n1)/c1cc2ccccc2[nH]1]

Its ¹H NMR (300 MHz, DMSO-d₆) exhibits peaks at δ 11.24 (d, J=2.2 Hz, 1H), 10.78 (d, J=31.5 Hz, 1H), 8.51 (dd, J=5.2, 1.8 Hz, 1H), 8.16 (d, J=16.6 Hz, 1H), 7.71 (td, J=7.6, 1.9 Hz, 1H), 7.52 (d, J=7.9 Hz, 1H), 7.40 (d, J=8.1 Hz, 1H), 7.36-7.18 (m, 2H), 7.12 (t, J=7.6 Hz, 1H), 7.03-6.90 (m, 2H), 6.67 (s, 1H), 3.82-3.55 (m, 10H), 3.00 (t, J=7.2 Hz, 2H).

The ¹³C NMR (151 MHz, DMSO-d₆) shows signals at δ 172.48, 166.16, 165.26, 164.57, 160.02, 149.51, 137.88, 136.90, 135.46, 134.58, 128.37, 123.68, 123.07, 121.90, 120.86, 119.83, 112.17, 104.72, 66.57, 43.75, 40.61, 31.43.

HRMS (ESI) gives m/z [M+H]⁺ calculated for C₂₃H₂₅N₉O: 444.2182, found: 444.2161.

### (E)-4-morpholino-N-(2-(pyridin-2-yl)ethyl)-6-(2-(quinolin-4-ylmethylene) hydrazineyl)-1,3,5-triazin-2-amine (AP5)

Compound AP5 is synthesized by compound 3a-4 and quinoline, resulting in a white solid with a 65% yield, and it has the following structure:

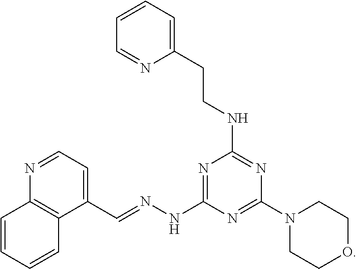 [SMILES: C(=N\Nc1nc(NCCc2ccccn2)nc(N2CCOCC2)n1)/c1ccnc2ccccc12]

Its ¹H NMR (300 MHz, DMSO-d6) exhibits peaks at δ 11.17 (d, J=104.2 Hz, 1H), 8.93 (t, J=3.3 Hz, 1H), 8.74-8.59 (m, 1H), 8.52 (dd, J=12.4, 4.9 Hz, 1H), 8.07 (t, J=8.4 Hz, 1H), 7.86-7.62 (m, 4H), 7.34-7.11 (m, 3H), 3.83-3.58 (m, 10H), 3.09-2.98 (m, 2H).

The ¹³C NMR (151 MHz, DMSO-d6) shows signals at δ 166.49, 165.27, 164.87, 159.98, 149.52, 136.90, 132.19, 132.06, 130.20, 129.91, 129.81, 129.14, 127.70, 125.05, 124.84, 123.66, 121.94, 121.88, 66.51, 43.74, 40.69, 37.91.

HRMS (ESI) gives m/z [M+H]⁺ calculated for C₂₄H₂₅N₉O: 456.2182, found: 446.2179.

### (E)-2-methoxy-4-((2-(4-morpholino-6-((2-(pyridin-2-yl)ethyl)amino)-1,3,5-triazin-2-yl) hydrazineylidene)methyl) phenol (AP6)

Compound AP6 is synthesized by compound 3a-4 and vanillin, resulting in a white solid with a 65% yield, and it has the following structure:

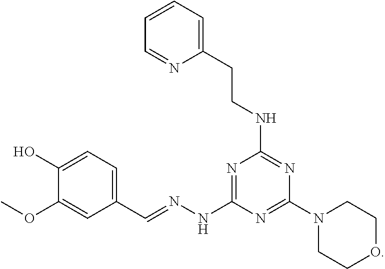 [SMILES: COc1cc(/C=N/Nc2nc(NCCc3ccccn3)nc(N3CCOCC3)n2)ccc1O]

Its ¹H NMR (300 MHz, DMSO-d6) exhibits peaks at δ 10.55 (d, J=46.8 Hz, 1H), 9.38 (s, 1H), 8.49 (d, J=4.9 Hz, 1H), 7.97 (d, J=8.3 Hz, 1H), 7.69 (td, J=7.7, 1.9 Hz, 1H), 7.37-6.95 (m, 5H), 6.79 (d, J=8.1 Hz, 1H), 3.82-3.54 (m, 13H), 2.97 (t, J=7.8 Hz, 2H).

The ¹³C NMR (151 MHz, DMSO-d6) shows signals at δ 166.24, 165.23, 164.65, 164.35, 160.03, 149.49, 148.40, 148.32, 136.85, 127.25, 127.10, 123.60, 121.85, 121.03, 115.96, 109.75, 66.54, 56.02, 43.68, 40.65, 38.00.

HRMS (ESI) gives m/z [M+H]⁺ calculated for C₂₂H₂₆N₈O₃: 451.2182, found: 446.2107.

### (E)-N-(cyclopropylmethyl)-4-(2-(3-iodobenzylidene) hydrazineyl)-6-morpholino-N-phenyl-1,3,5-triazin-2-amine (DP1)

Compound DP1 is synthesized by compound 3a-9 and 3-iodobenzaldehyde, resulting in a white solid with a 78% yield, and it has the following structure:

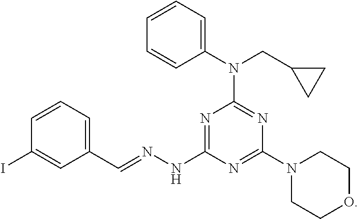 [SMILES: Ic1cccc(/C=N/Nc2nc(N3CCOCC3)nc(N(CC3CC3)c3ccccc3)n2)c1]

Its ¹H NMR (600 MHz, DMSO-d6) exhibits peaks at δ 10.88 (s, 1H), 7.97 (d, J=29.4 Hz, 2H), 7.69 (dt, J=7.8, 1.3 Hz, 1H), 7.60 (d, J=32.8 Hz, 1H), 7.41 (t, J=7.7 Hz, 2H), 7.33 (d, J=7.8 Hz, 2H), 7.26 (t, J=7.4 Hz, 1H), 7.21-7.15 (m, 1H), 3.85 (s, 2H), 3.60 (s, 8H), 1.09 (s, 1H), 0.42-0.38 (m, 2H), 0.19 (s, 2H).

The ¹³C NMR (151 MHz, DMSO-d6) shows signals at δ 165.73, 165.19, 164.60, 144.10, 140.24, 138.07, 137.71, 137.42, 131.27, 129.01, 128.52, 126.28, 122.80, 120.48, 95.66, 66.45, 54.03, 43.70, 10.71, 3.97, 3.95.

HRMS (ESI) gives m/z [M+H]⁺ calculated for C₂₄H₂₆IN₇O: 556.1244, found: 556.1194.

### (E)-4-(2-((1H-indol-3-yl)methylene) hydrazineyl)-N-(cyclopropylmethyl)-6-morpholino-N-phenyl-1,3,5-triazin-2-amine (DP2)

Compound DP2 is synthesized by compound 3a-9 and indole-3-carboxaldehyde, resulting in a white solid with a 77% yield, and it has the following structure:

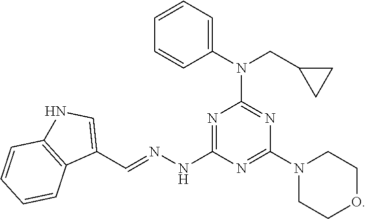 [SMILES: C(=N\Nc1nc(N2CCOCC2)nc(N(CC2CC2)c2ccccc2)n1)/c1c[nH]c2ccccc12]

Its ¹H NMR (600 MHz, DMSO-d6) exhibits peaks at δ 11.35 (s, 1H), 10.48 (s, 1H), 8.24 (s, 1H), 7.63 (s, 1H), 7.46-7.23 (m, 6H), 7.17 (t, J=7.6 Hz, 1H), 7.06 (s, 1H), 3.78 (d, J=206.6 Hz, 10H), 1.18 (t, J=7.1 Hz, 1H), 0.41 (d, J=7.8 Hz, 2H), 0.19 (d, J=82.8 Hz, 2H).

The ¹³C NMR (151 MHz, DMSO-d6) shows signals at δ 165.92, 165.19, 164.46, 144.35, 139.41, 137.42, 128.95, 128.61, 126.06, 123.08, 122.80, 120.48, 113.10, 111.93, 66.47, 60.23, 54.03, 43.71, 10.80, 3.95.

HRMS (ESI) gives m/z [M+H]⁺ calculated for C₂₆H₂₈N₈O: 469.2386, found: 469.2350.

### (E)-4-((2-(4-((cyclopropylmethyl) (phenyl)amino)-6-morpholino-1,3,5-triazin-2-yl) hydrazineylidene)methyl)-2-methoxyphenol (DP3)

Compound DP3 is synthesized by compound 3a-9 and vanillin, resulting in a white solid with a 68% yield, and it has the following structure:

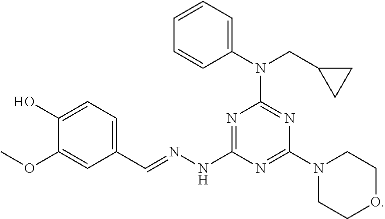 [SMILES: COc1cc(/C=N/Nc2nc(N3CCOCC3)nc(N(CC3CC3)c3ccccc3)n2)ccc1O]

Its ¹H NMR (600 MHz, DMSO-d6) exhibits peaks at δ 10.59 (s, 1H), 9.35 (s, 1H), 7.96 (s, 1H), 7.41-7.37 (m, 2H), 7.32 (d, J=7.7 Hz, 2H), 7.25-7.15 (m, 2H), 6.93 (s, 1H), 6.78 (d, J=8.0 Hz, 1H), 3.84 (s, 2H), 3.79 (s, 3H), 3.60 (s, 8H), 1.11-1.05 (m, 1H), 0.41-0.37 (m, 2H), 0.18 (s, 2H).

The ¹³C NMR (151 MHz, DMSO-d6) shows signals at δ 165.78, 165.16, 164.52, 148.38, 148.34, 144.27, 142.50, 128.96, 128.58, 127.22, 126.09, 115.81, 66.47, 55.81, 54.07, 43.68, 10.71, 3.95.

HRMS (ESI) gives m/z [M+H]⁺ calculated for C₂₅H₂₉N₇O₃: 476.2332, found: 476.2299.

### (E)-2-((2-(4-((cyclopropylmethyl) (phenyl)amino)-6-morpholino-1,3,5-triazin-2-yl) hydrazineylidene)methyl)-4-iodophenol (DP4)

Compound DP4 is synthesized by compound 3a-9 and 2-hydroxy-5-methylbenzaldehyde, resulting in a white solid with a 65% yield, and it has the following structure:

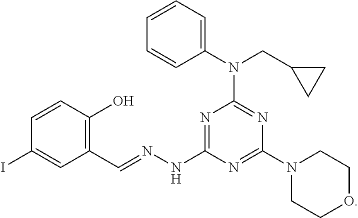 [SMILES: Oc1ccc(I)cc1/C=N/Nc1nc(N2CCOCC2)nc(N(CC2CC2)c2ccccc2)n1]

Its ¹H NMR (600 MHz, DMSO-d6) exhibits peaks at & 12.22 (s, 1H), 11.30 (s, 1H), 8.15 (s, 1H), 7.68 (s, 1H), 7.49 (dd, J=8.6, 2.3 Hz, 1H), 7.45-7.37 (m, 2H), 7.31 (d, J=8.2 Hz, 2H), 7.26 (tt, J=7.2, 1.3 Hz, 1H), 6.74 (d, J=8.7 Hz, 1H), 3.81 (s, 2H), 3.61 (s, 6H), 1.06 (s, 1H), 0.44-0.34 (m, 2H), 0.12 (s, 2H).

The ¹³C NMR (151 MHz, DMSO-d6) shows signals at δ 165.69, 164.99, 164.13, 157.51, 143.97, 142.17, 138.55, 137.70, 129.16, 128.64, 126.47, 122.11, 119.53, 81.21, 66.36, 43.65, 10.64, 3.93.

HRMS (ESI) gives m/z [M+H]⁺ calculated for C₂₄H₂₆IN₇O₂: 572.1193, found: 572.1202.

### E)-4-(2,3-dihydro-4H-benzo[b][1,4]oxazin-4-yl)-N-(3-iodostyryl)-6-morpholino-1,3,5-triazin-2-amine (G1)

Compound G1 is synthesized by compound 3a-6 and 3-iodobenzaldehyde, resulting in a white solid with a 71% yield, and it has the following structure:

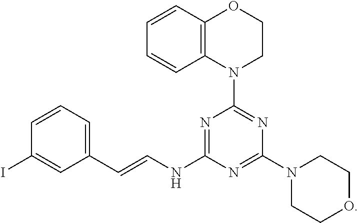 [SMILES: Ic1cccc(/C=C/Nc2nc(N3CCOCC3)nc(N3CCOc4ccccc43)n2)c1]

Its ¹H NMR (600 MHz, DMSO-d6) exhibits peaks at δ 11.19 (s, 1H), 8.05 (d, J=12.0 Hz, 3H), 7.73 (dt, J=8.0, 1.2 Hz, 1H), 7.65 (d, J=7.8 Hz, 1H), 7.23 (t, J=7.8 Hz, 1H), 7.08-6.82 (m, 3H), 4.35-4.15 (m, 4H), 3.86-3.58 (m, 8H).

The ¹³C NMR (151 MHz, DMSO-d6) shows signals at δ 165.17, 164.63, 164.08, 146.61, 137.98, 137.86, 131.34, 127.39, 125.80, 124.38, 119.78, 116.98, 95.76, 66.44, 65.97, 43.85, 41.41.

HRMS (ESI) gives m/z [M+H]⁺ calculated for C₂₃H₂₃IN₆O₂: 543.0927, found: 544.0839.

### (E)-4-(4-(2-((1H-indol-3-yl)methylene) hydrazineyl)-6-morpholino-1,3,5-triazin-2-yl)-3,4-dihydro-2H-benzo[b][1,4]oxazine (G2)

Compound G2 is synthesized by compound 3a-6 and indole-3-carboxaldehyde, resulting in a white solid with a 72% yield, and it has the following structure:

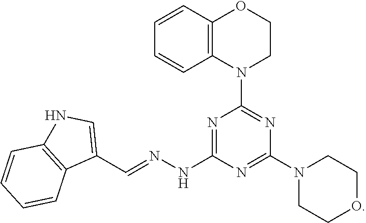 [SMILES: C(=N\Nc1nc(N2CCOCC2)nc(N2CCOc3ccccc32)n1)/c1c[nH]c2ccccc12]

Its ¹H NMR (600 MHz, DMSO-d6) exhibits peaks at δ 11.44 (s, 1H), 10.74 (s, 1H), 8.35 (d, J=24.6 Hz, 3H), 7.70 (d, J=2.6 Hz, 1H), 7.45-7.38 (m, 1H), 7.20 (t, J=7.5 Hz, 1H), 7.10 (td, J=7.4, 1.1 Hz, 1H), 7.06-6.84 (m, 3H), 4.29 (s, 4H), 3.70 (d, J=18.8 Hz, 8H).

The ¹³C NMR (151 MHz, DMSO-d6) shows signals at δ 164.37, 146.51, 140.63, 137.47, 129.61, 127.75, 125.70, 124.11, 122.91, 122.87, 120.61, 119.83, 116.93, 112.82, 112.07, 66.49, 66.03, 43.84, 41.48.

HRMS (ESI) gives m/z [M+H]⁺ calculated for C₂₄H₂₄N₈O₂: 457.2022, found: 457.1992.

### (E)-4-(4-(3,4-dihydroquinolin-1 (2H)-yl)-6-(2-(3-iodobenzylidene)hydrazineyl)-1,3,5-triazin-2-yl)morpholine (K1)

Compound K₁ is synthesized by compound 3a-7 and 3-iodobenzaldehyde, resulting in a white solid with a 78% yield, and it has the following structure:

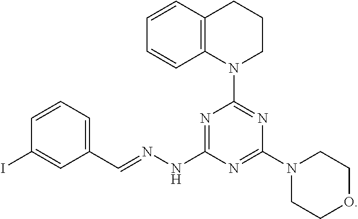 [SMILES: Ic1cccc(/C=N/Nc2nc(N3CCOCC3)nc(N3CCCc4ccccc43)n2)c1]

Its ¹H NMR (300 MHz, DMSO-d₆) exhibits peaks at δ 11.06 (s, 1H), 8.02 (d, J=7.1 Hz, 3H), 7.71 (dt, J=8.0, 1.2 Hz, 1H), 7.62 (d, J=7.7 Hz, 1H), 7.22 (t, J=7.8 Hz, 2H), 7.14 (dd, J=7.6, 1.6 Hz, 1H), 7.02 (t, J=7.2 Hz, 1H), 4.01 (t, J=5.9 Hz, 2H), 3.79-3.59 (m, 8H), 2.77 (t, J=6.6 Hz, 2H), 1.90 (p, J=6.5 Hz, 2H).

The ¹³C NMR (151 MHz, DMSO-d₆) shows signals at δ 165.24, 164.96, 164.58, 140.43, 139.59, 137.94, 137.84, 131.31, 130.93, 128.79, 126.37, 125.24, 123.50, 95.72, 66.46, 44.17, 43.80, 27.29, 23.77.

HRMS (ESI) gives m/z [M+H]⁺ calculated for C₂₃H₂₄IN₇O: 542.1087, found: 542.1044.

### (E)-4-(4-(2-((1H-indol-3-yl)methylene) hydrazineyl)-6-(3,4-dihydroquinolin-1 (2H)-yl)-1,3,5-triazin-2-yl) morpholine (K2)

Compound K2 is synthesized by compound 3a-7 and indole-3-carboxaldehyde, resulting in a white solid with a 67% yield, and it has the following structure:

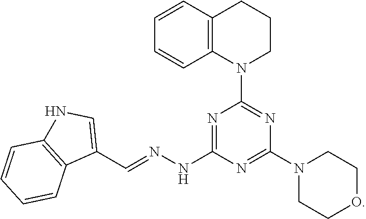 [SMILES: C(=N\Nc1nc(N2CCOCC2)nc(N2CCCc3ccccc32)n1)/c1c[nH]c2ccccc12]

Its ¹H NMR (300 MHz, DMSO-d6) exhibits peaks at δ 11.41 (d, J=2.7 Hz, 1H), 10.63 (s, 1H), 8.31 (d, J=16.7 Hz, 3H), 7.67 (d, J=2.6 Hz, 1H), 7.40 (d, J=8.0 Hz, 1H), 7.22-7.12 (m, 3H), 7.08 (t, J=7.4 Hz, 2H), 4.14-3.98 (m, 2H), 3.68 (d, J=13.7 Hz, 8H), 2.79 (t, J=6.7 Hz, 2H), 1.91 (d, J=7.9 Hz, 2H).

The ¹³C NMR (151 MHz, DMSO-d6) shows signals at δ 165.17, 164.41, 139.89, 137.44, 130.77, 129.27, 128.81, 126.26, 125.32, 124.83, 123.18, 122.87, 120.50, 112.96, 112.01, 66.51, 44.22, 43.80, 27.32, 23.85.

HRMS (ESI) gives m/z [M+H]⁺ calculated for C₂₅H₂₆N₈O: 455.2230, found: 455.2179.

### (E)-4-((2-(4-(3,4-dihydroquinolin-1 (2H)-yl)-6-morpholino-1,3,5-triazin-2-yl) hydrazineylidene)methyl)-2-methoxyphenol (K3)

Compound K3 is synthesized by compound 3a-7 and vanillin, resulting a white solid with a 66% yield, and it has the following structure:

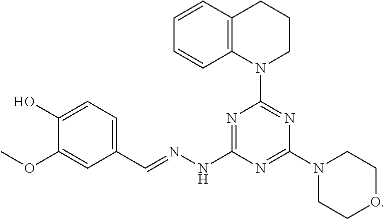 [SMILES: COc1cc(/C=N/Nc2nc(N3CCOCC3)nc(N3CCCc4ccccc43)n2)ccc1O]

Its ¹H NMR (300 MHz, DMSO-d6) exhibits peaks at δ 10.76 (s, 1H), 9.40 (s, 1H), 7.98 (s, 2H), 7.22 (d, J=1.8 Hz, 1H), 7.13 (ddt, J=7.3, 4.8, 2.4 Hz, 2H), 6.98 (ddd, J=9.2, 5.8, 2.1 Hz, 2H), 6.80 (d, J=8.1 Hz, 1H), 4.00 (t, J=5.9 Hz, 2H), 3.80 (s, 3H), 3.76-3.56 (m, 8H), 2.77 (t, J=6.7 Hz, 2H), 1.95-1.84 (m, 2H).

The ¹³C NMR (151 MHz, DMSO-d6) shows signals at & 165.21, 165.02, 164.49, 148.51, 148.35, 142.91, 139.75, 130.88, 128.81, 127.08, 126.31, 125.08, 123.29, 121.26, 115.91, 66.49, 55.92, 44.13, 43.78, 27.25, 23.81.

HRMS (ESI) gives m/z [M+H]⁺ calculated for C₂₄H₂₇N₇O₃: 462.2175, found: 462.2145.

### (E)-4-(2-(3-iodobenzylidene) hydrazineyl)-N-(2-(pyridin-2-yl)ethyl)-6-thiomorpholino-1,3,5-triazin-2-amine (APS1)

Compound APS1 is synthesized by compound 3b and 3-Iodobenzaldehyde, resulting in a white solid with a 78% yield, and it has the following structure:

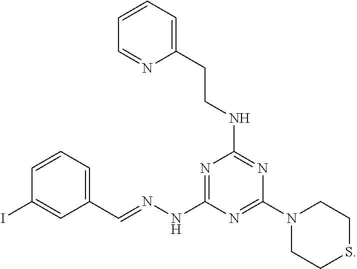 [SMILES: Ic1cccc(/C=N/Nc2nc(NCCc3ccccn3)nc(N3CCSCC3)n2)c1]

Its ¹H NMR (600 MHz, DMSO-d6) exhibits peaks at δ 10.90 (s, 1H), 8.51 (d, J=5.1 Hz, 1H), 8.08-7.97 (m, 2H), 7.77-7.69 (m, 2H), 7.62 (dd, J=35.1, 7.8 Hz, 1H), 7.37-7.05 (m, 4H), 4.03 (dd, J=6.7, 3.3 Hz, 4H), 3.62 (dq, J=13.6, 6.7 Hz, 2H), 3.00 (q, J=7.3 Hz, 2H), 2.61 (q, J=8.8, 7.2 Hz, 4H).

The ¹³C NMR (151 MHz, DMSO-d6) shows signals at δ 164.77, 159.90, 149.49, 140.20, 138.08, 137.80, 136.96, 134.97, 134.48, 131.32, 126.62, 126.11, 123.65, 123.58, 121.91, 95.69, 45.75, 40.69, 31.43, 22.53.

HRMS (ESI) gives m/z [M+H]⁺ calculated for C₂₁H₂₃IN₈S: 547.0811, found: 547.0788.

### (E)-4-(2-((1H-indol-3-yl)methylene) hydrazineyl)-N-(2-(pyridin-2-yl)ethyl)-6-thiomorpholino-1,3,5-triazin-2-amine (APS2)

Compound APS2 is synthesized by compound 3b and indole-3-carboxaldehyde, resulting in a white solid with a 70% yield, and it has the following structure:

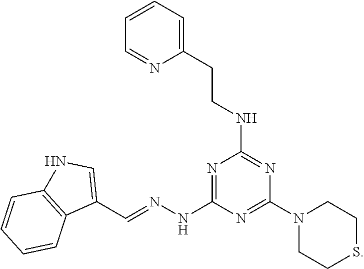 [SMILES: C(=N\Nc1nc(NCCc2ccccn2)nc(N2CCSCC2)n1)/c1c[nH]c2ccccc12]

Its ¹H NMR (600 MHz, DMSO-d6) exhibits peaks at δ 11.37 (d, J=33.1 Hz, 1H), 10.45 (d, J=81.4 Hz, 1H), 8.56-8.50 (m, 1H), 8.49-8.33 (m, 1H), 8.27 (s, 1H), 7.75-7.62 (m, 2H), 7.40 (dd, J=13.2, 7.9 Hz, 1H), 7.30 (dd, J=21.3, 7.6 Hz, 1H), 7.23 (d, J=6.2 Hz, 1H), 7.15 (dt, J=41.3, 7.5 Hz, 2H), 7.03-6.67 (m, 1H), 4.08 (s, 4H), 3.84-3.58 (m, 2H), 3.02 (t, J=7.1 Hz, 2H), 2.64 (s, 4H).

The ¹³C NMR (151 MHz, DMSO-d6) shows signals at δ 172.47, 164.69, 160.02, 149.51, 139.00, 137.41, 136.91, 128.56, 124.97, 123.63, 122.99, 122.76, 121.88, 120.40, 113.19, 112.06, 45.56, 40.68, 31.43, 26.83.

HRMS (ESI) gives m/z [M+H]⁺ calculated for C₂₃H₂₅N₉S: 460.1954, found 460.1938.

### (E)-4-(2-(3-iodobenzylidene) hydrazineyl)-6-morpholino-N-(pyridin-2-ylmethyl)-1,3,5-triazin-2-amine (T1)

Compound Tl is synthesized by compound 3a-5 and 3-iodobenzaldehyde, resulting in a white solid with a 70% yield, and it has the following structure:

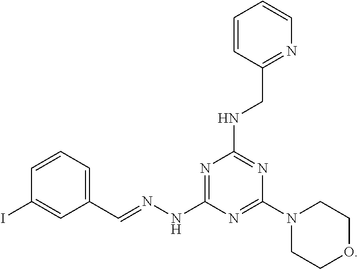 [SMILES: Ic1cccc(/C=N/Nc2nc(NCc3ccccn3)nc(N3CCOCC3)n2)c1]

Its ¹H NMR (600 MHz, DMSO-d6) exhibits peaks at δ 10.85 (d, J=8.3 Hz, 1H), 8.62-8.46 (m, 1H), 8.08-7.93 (m, 2H), 7.76-7.68 (m, 2H), 7.64-7.52 (m, 1H), 7.32 (d, J=7.9 Hz, 1H), 7.23 (q, J=8.1, 7.1 Hz, 2H), 4.58 (dd, J=31.6, 6.2 Hz, 2H), 3.63 (t, J=51.8 Hz, 8H).

The ¹³C NMR (151 MHz, DMSO-d6) shows signals at δ 166.64, 166.50, 165.31, 165.15, 164.63, 160.18, 160.06, 149.22, 149.10, 140.40, 137.92, 137.75, 136.96, 134.99, 134.52, 131.32, 126.53, 126.07, 122.34, 122.26, 121.31, 95.68, 66.53, 66.39, 46.20, 43.66.

HRMS (ESI) gives m/z [M+H]⁺ calculated for C₂₀H₂₁IN₈O: 517.0883, found: 517.0856.

### (E)-4-(2-((1H-indol-3-yl)methylene) hydrazineyl)-6-morpholino-N-(pyridin-2-ylmethyl)-1,3,5-triazin-2-amine (T2)

Compound T2 is synthesized by compound 3a-5 and indole-3-carboxaldehyde, resulting in a white solid with a 72% yield, and it has the following structure:

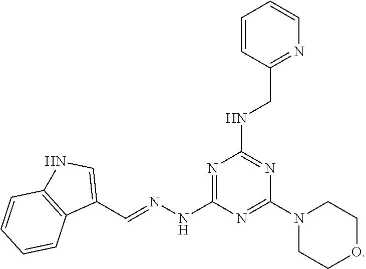 [SMILES: C(=N\Nc1nc(NCc2ccccn2)nc(N2CCOCC2)n1)/c1c[nH]c2ccccc12]

Its ¹H NMR (600 MHz, DMSO-d6) exhibits peaks at δ 11.36 (d, J=28.2 Hz, 1H), 10.46 (d, J=56.4 Hz, 1H), 8.51 (t, J=10.4 Hz, 1H), 8.40 (s, 1H), 8.31-8.20 (m, 1H), 7.74 (t, J=7.3 Hz, 1H), 7.64 (d, J=19.0 Hz, 1H), 7.52-7.29 (m, 3H), 7.21-7.02 (m, 2H), 4.91-4.51 (m, 2H), 3.62 (d, J=54.2 Hz, 8H).

The ¹³C NMR (151 MHz, DMSO-d6) shows signals at δ 165.42, 164.62, 162.78, 160.41, 149.18, 149.09, 139.14, 137.39, 136.97, 128.60, 124.94, 122.80, 122.24, 121.41, 121.13, 120.44, 113.12, 112.02, 66.56, 66.46, 46.20, 45.84, 43.73.

HRMS (ESI) gives m/z [M+H]⁺ calculated for C₂₂H₂₃N₉O: 430.2026, found: 430.2020.

### E)-4-(4-(indolin-1-yl)-6-(2-(3-iodobenzylidene)hydrazineyl)-1,3,5-triazin-2-yl)morpholine (L1)

Compound L1 is synthesized by compound 3a-8 and 3-iodobenzaldehyde, resulting in a white solid with a 75% yield, and it has the following structure:

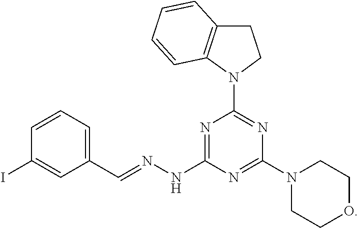 [SMILES: Ic1cccc(/C=N/Nc2nc(N3CCOCC3)nc(N3CCc4ccccc43)n2)c1]

Its ¹H NMR (600 MHz, DMSO-d6) exhibits peaks at δ 11.19 (s, 1H), 8.06 (s, 3H), 7.74 (dt, J=8.0, 1.3 Hz, 1H), 7.67 (s, 1H), 7.46-7.21 (m, 3H), 6.95 (t, J=7.4 Hz, 1H), 4.16 (s, 2H), 3.73 (d, J=61.6 Hz, 8H), 3.14 (t, J=8.6 Hz, 2H).

The ¹³C NMR (151 MHz, DMSO-d6) shows signals at δ 165.05, 164.38, 163.39, 143.64, 137.98, 137.89, 133.01, 131.42, 127.35, 125.17, 122.24, 95.84, 66.47, 43.85, 27.03.

HRMS (ESI) gives m/z [M+H]⁺ calculated for C₂₂H₂₂IN₇O: 528.0931, found: 528.0883.

### (E)-4-(4-(2-((1H-indol-3-yl)methylene) hydrazineyl)-6-(indolin-1-yl)-1,3,5-triazin-2-yl) morpholine (L2)

Compound L2 is synthesized by compound 3a-8 and indole-3-carboxaldehyde, resulting a white solid with a 77% yield, and it has the following structure:

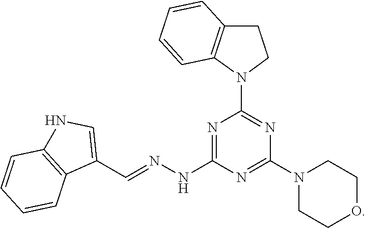 [SMILES: C(=N\Nc1nc(N2CCOCC2)nc(N2CCc3ccccc32)n1)/c1c[nH]c2ccccc12]

Its ¹H NMR (600 MHz, DMSO-d6) exhibits peaks at δ 11.46 (d, J=41.9 Hz, 1H), 10.74 (d, J=48.3 Hz, 1H), 8.98 (d, J=75.7 Hz, 1H), 8.57 (s, 1H), 8.35 (d, J=5.6 Hz, 1H), 7.74 (s, 1H), 7.47 (dd, J=15.7, 8.1 Hz, 1H), 7.31-7.17 (m, 4H), 6.96 (d, J=31.6 Hz, 1H), 4.17 (s, 2H), 3.89-3.61 (m, 8H), 3.15 (s, 2H).

The ¹³C NMR (151 MHz, DMSO-d6) shows signals at δ 164.90, 164.12, 163.68, 155.51, 143.87, 140.87, 137.69, 137.59, 132.93, 129.95, 127.12, 125.19, 125.11, 124.77, 123.14, 122.98, 122.55, 122.09, 121.06, 120.65, 117.46, 112.82, 112.44, 112.21, 66.52, 48.04, 43.83, 27.02.

HRMS (ESI) gives m/z [M+H]⁺ calculated for C₂₄H₂₄N₈O: 441.2073, found: 441.2053.

### (E)-4-([1,4′-bipiperidin]-1′-yl)-N-(3-fluorobenzyl)-6-(2-(3-(trifluoromethyl)benzylidene)hydrazineyl)-1,3,5-triazin-2-amine (E1)

Compound E1 is synthesized by compound 3c and 3-(Trifluoromethyl)benzaldehyde, resulting in a white solid with a 65% yield, and it has the following structure:

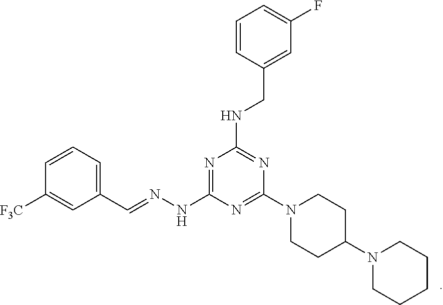 [SMILES: Fc1cccc(CNc2nc(N/N=C/c3cccc(C(F)(F)F)c3)nc(N3CCC(N4CCCCC4)CC3)n2)c1]

Its ¹H NMR (600 MHz, DMSO-d6) exhibits peaks at δ 10.94 (d, J=40.8 Hz, 1H), 8.16 (d, J=3.8 Hz, 1H), 7.99 (d, J=22.4 Hz, 1H), 7.91 (dd, J=14.2, 7.7 Hz, 1H), 7.78-7.53 (m, 3H), 7.35 (q, J=7.4 Hz, 1H), 7.26-7.11 (m, 2H), 7.04 (td, J=8.7, 2.7 Hz, 1H), 4.71 (s, 2H), 4.47 (dd, J=27.3, 6.3 Hz, 2H), 2.89-2.52 (m, 6H), 1.86 (s, 2H), 1.60 (s, 4H), 1.45-1.18 (m, 5H).

The ¹³C NMR (151 MHz, DMSO-d6) shows signals at & 166.66, 166.49, 164.76, 163.43, 161.82, 144.24, 144.20, 140.25, 140.03, 136.91, 136.81, 130.78, 130.49, 130.44, 130.35, 130.10, 129.90, 125.55, 125.50, 123.81, 123.69, 122.60, 114.51, 114.37, 113.75, 113.61, 62.71, 49.90, 43.72, 42.45, 31.43, 22.53, 14.43.

HRMS (ESI) gives m/z [M+H]⁺ calculated for C₂₈H₃₂F₄N₈: 557.2688, found: 557.2656.

### (E)-4-(6,6-dimethyl-3-azabicyclo[3.1.0]hexan-3-yl)-N-(3-fluorobenzyl)-6-(2-(quinolin-4-ylmethylene) hydrazineyl)-1,3,5-triazin-2-amine (F1)

Compound F1 is synthesized by compound 3d and 4-quinolinecarboxaldehyde, resulting in a white solid with a 65% yield, and it has the following structure:

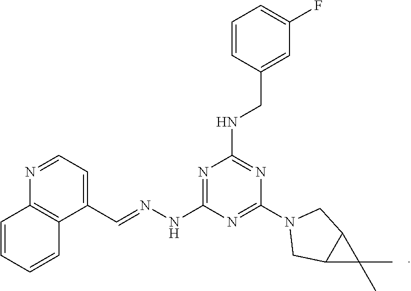 [SMILES: CC1(C)C2CN(c3nc(NCc4cccc(F)c4)nc(N/N=C/c4ccnc5ccccc45)n3)CC21]

Its ¹H NMR (600 MHz, DMSO-d6) exhibits peaks at δ 11.22 (s, 1H), 9.27-8.83 (m, 2H), 8.68 (d, J=79.2 Hz, 1H), 8.07 (t, J=9.1 Hz, 1H), 7.86-7.62 (m, 4H), 7.35 (t, J=7.1 Hz, 1H), 7.19 (dt, J=21.5, 12.8 Hz, 2H), 7.09-7.00 (m, 1H), 4.70-4.37 (m, 2H), 3.52 (d, J=2.5 Hz, 2H), 1.53-1.41 (m, 2H), 1.04 (d, J=5.9 Hz, 3H), 0.84 (d, J=27.5 Hz, 3H).

The ¹³C NMR (151 MHz, DMSO-d6) shows signals at δ 166.32, 165.55, 164.39, 163.43, 163.24, 162.91, 161.81, 150.75, 150.68, 148.91, 144.16, 144.03, 139.48, 138.65, 138.56, 130.50, 130.43, 130.13, 129.93, 129.84, 127.74, 127.61, 126.09, 125.10, 124.86, 124.05, 120.68, 114.73, 114.59, 113.84, 113.70, 46.35, 43.73, 43.41, 27.28, 27.07, 26.45, 19.12, 12.82, 12.73.

HRMS (ESI) gives m/z [M+H]⁺ calculated for C₂₇H₂₇FN₈: 483.2343, found: 483.2322.

### (E)-4-(6,6-dimethyl-3-azabicyclo[3.1.0]hexan-3-yl)-N-(3-fluorobenzyl)-6-(2-(3-iodobenzylidene) hydrazineyl)-1,3,5-triazin-2-amine (F2)

Compound F2 is synthesized by compound 3d and 3-iodobenzaldehyde, resulting in a white solid with a 68% yield, and it has the following structure:

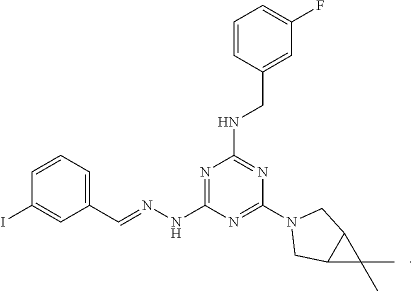 [SMILES: CC1(C)C2CN(c3nc(NCc4cccc(F)c4)nc(N/N=C/c4cccc(I)c4)n3)CC21]

Its ¹H NMR (600 MHz, DMSO-d6) exhibits peaks at δ 10.86 (s, 1H), 8.02 (t, J=10.7 Hz, 2H), 7.85-7.50 (m, 3H), 7.35 (td, J=7.9, 6.1 Hz, 1H), 7.31-7.09 (m, 3H), 7.09-7.00 (m, 1H), 4.56-4.36 (m, 2H), 3.51 (q, J=12.1, 7.9 Hz, 4H), 1.44 (d, J=4.6 Hz, 2H), 1.03 (s, 3H), 0.87-0.76 (m, 3H).

The ¹³C NMR (151 MHz, DMSO-d6) shows signals at δ 164.23, 163.40, 161.80, 144.14, 139.98, 138.15, 137.82, 134.78, 134.41, 131.32, 130.47, 130.41, 126.74, 126.21, 124.05, 114.74, 114.60, 113.82, 113.69, 95.70, 46.39, 46.31, 43.70, 27.27, 27.00, 26.44, 19.08, 12.82, 12.73.

HRMS (ESI) gives m/z [M+H]⁺ calculated for C₂₄H₂₅FIN₇: 558.1200, found: 559.1116.

### (E)-4-(6,6-dimethyl-3-azabicyclo[3.1.0]hexan-3-yl)-N-(3-fluorobenzyl)-6-(2-(3-(trifluoromethyl)benzylidene) hydrazineyl)-1,3,5-triazin-2-amine (F3)

Compound F3 is synthesized by compound 3d and 3-(trifluoromethyl)benzaldehyde, resulting in a white solid with a 71% yield, and it has the following structure:

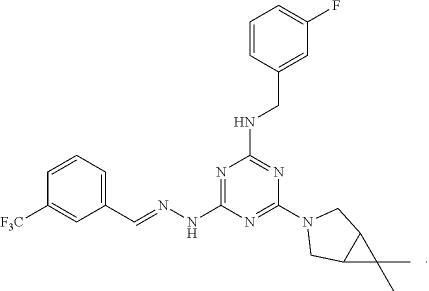 [SMILES: CC1(C)C2CN(c3nc(NCc4cccc(F)c4)nc(N/N=C/c4cccc(C(F)(F)F)c4)n3)CC21]

Its ¹H NMR (600 MHz, DMSO-d6) exhibits peaks at δ 10.97 (s, 1H), 8.17 (d, J=13.4 Hz, 1H), 7.99 (d, J=10.6 Hz, 1H), 7.91 (d, J=7.7 Hz, 1H), 7.83-7.52 (m, 3H), 7.34 (dq, J=15.8, 7.2, 6.7 Hz, 1H), 7.18 (dt, J=20.5, 12.9 Hz, 2H), 7.08-6.99 (m, 1H), 4.56-4.38 (m, 2H), 3.62-3.41 (m, 4H), 1.45 (d, J=4.5 Hz, 2H), 1.03 (s, 3H), 0.83 (d, J=20.9 Hz, 3H).

The ¹³C NMR (151 MHz, DMSO-d6) shows signals at δ 166.14, 164.27, 163.40, 163.19, 161.79, 144.13, 139.93, 136.97, 136.63, 130.93, 130.62, 130.47, 130.41, 130.34, 130.11, 129.90, 129.69, 127.29, 125.71, 125.49, 124.05, 123.68, 122.67, 121.88, 114.74, 114.60, 113.82, 113.69, 46.32, 43.70, 27.26, 27.00, 26.43, 19.08, 12.72.

HRMS (ESI) gives m/z [M+H]⁺ calculated for C₂₅H₂₅F₄N₇: 500.2108, found: 500.20.

### methyl (E)-3-((4-(2-(3-iodobenzylidene) hydrazineyl)-6-morpholino-1,3,5-triazin-2-yl) (phenyl)amino) propanoate (A1)

Compound A1 is synthesized by compound 3d and 3-iodobenzaldehyde, resulting in a white solid with a 73% yield, and it has the following structure:

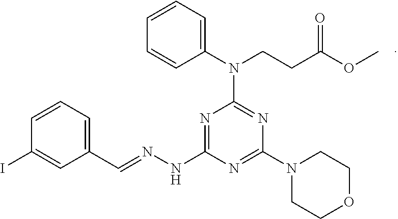 [SMILES: COC(=O)CCN(c1ccccc1)c1nc(N/N=C/c2cccc(I)c2)nc(N2CCOCC2)n1]

Its ¹H NMR (600 MHz, DMSO-d6) exhibits peaks at δ 10.91 (s, 1H), 8.00 (s, 1H), 7.92 (s, 1H), 7.69 (d, J=7.8 Hz, 1H), 7.58 (s, 1H), 7.42 (t, J=7.7 Hz, 2H), 7.33 (d, J=7.8 Hz, 2H), 7.27 (t, J=7.5 Hz, 1H), 7.19 (t, J=7.8 Hz, 1H), 4.19 (s, 2H), 3.57 (d, J=59.2 Hz, 11H), 2.66 (s, 2H).

The ¹³C NMR (151 MHz, DMSO-d6) shows signals at δ 172.14, 165.53, 165.12, 164.57, 143.45, 140.50, 137.98, 137.79, 134.87, 131.25, 129.10, 128.33, 126.47, 126.22, 95.64, 66.44, 51.82, 46.38, 43.69, 33.04.

High-resolution mass spectrometry (HRMS, ESI) gives m/z [M+H]⁺ calculated for C₂₄H₂₆IN₇O₃ as 588.1142, found: 588.1043.

### methyl (E)-3-((4-morpholino-6-(2-(quinolin-4-ylmethylene) hydrazineyl)-1,3,5-triazin-2-yl) (phenyl)amino) propanoate (A2)

Compound A2 is synthesized by compound 3d and 4-quinolinecarboxaldehyde, resulting in a white solid with a 66% yield, and it has the following structure:

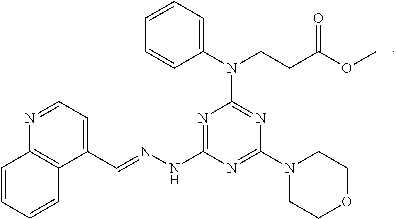 [SMILES: COC(=O)CCN(c1ccccc1)c1nc(N/N=C/c2ccnc3ccccc23)nc(N2CCOCC2)n1]

Its ¹H NMR (600 MHz, DMSO-d6) exhibits peaks at δ 11.26 (s, 1H), 9.07 (s, 1H), 8.93 (d, J=4.6 Hz, 1H), 8.62 (s, 1H), 8.07 (d, J=8.4 Hz, 1H), 7.81 (t, J=7.6 Hz, 1H), 7.64 (d, J=24.8 Hz, 2H), 7.44 (t, J=7.6 Hz, 2H), 7.38-7.27 (m, 3H), 4.22 (s, 2H), 3.74 (d, J=103.7 Hz, 8H), 3.51 (s, 3H), 2.69 (s, 2H).

The ¹³C NMR (151 MHz, DMSO-d6) shows signals at δ 172.15, 165.72, 165.10, 164.70, 150.54, 148.59, 143.43, 138.67, 130.03, 129.86, 129.25, 128.45, 127.87, 126.63, 125.91, 124.83, 66.43, 51.81, 46.54, 43.72, 32.99.

HRMS (ESI) gives m/z [M+H]⁺ calculated for C₂₇H₂₈N₈O₃: 513.2284, found: 513.2216.

The 1,3,5-triazine derivatives or the present invention can inhibit endosomal trafficking and autophagy in vitro and inhibit tumor growth and metastasis of malignant tumor cells in vivo.

Among them, two hit compounds, AP1 and AP2, are more potent than others in inhibiting endosomal trafficking and autophagy. Systematic studies are conducted on the in vitro and in vivo antitumor activity of AP1 and AP2. AP1 and AP2 inhibit cancer cell proliferation, growth, and migration, arrest the cell cycle at the G0/G1 phase, induce cell death via non-apoptotic pathways, and inhibit tumor sphere formation.

Moreover, AP1 potently inhibits tumor growth and metastasis of various cancer cells, including lung cancer cells and melanoma cells, in different mouse models, with minimal toxicity observed in mice at the given doses. AP1 also regulates macrophage polarization and the tumor microenvironment by modulating cytokine secretion.

In the present invention, the addition of a morpholino group at the 6-position is crucial; substituting it with other groups results in a loss of activity. Secondly, mono-substitution with side chains of varying lengths at the 2-position maintains potency. Among them, the 2-carbon length shows superior efficacy, and incorporating heterocyclic groups containing a nitrogen atom may further enhance activity. Thirdly, altering the para-position of 3-iodobenzene is not suitable, resulting in a loss of potency. Similarly, the incorporation of a nitrogenous heterocyclic group shows superiority over other groups, although it is still not matching the potency of the 3-iodobenzene group.

As used herein, “pharmaceutically acceptable carrier” is intended to mean a non-toxic solid, semisolid or liquid filer, diluent, encapsulating material or formulation auxiliary of any type. The modes of administration which include intravenous, intraperitoneal, intramuscular, intrasternal, subcutaneous and intraarticular injection and infusion. It is obvious for one skilled person in the art to recognize that the choice of a particular mode of administration can be made based upon considerations such as the particular disease state being treated; the type and degree of the response to be achieved; the specific agent or composition employed; the time of administration and rate or excretion of the agent or composition; the age, body weight, general health, sex and diet of the patient; the duration of the treatment; drugs (such as a chemotherapeutic agent) used in combination or coincidental with the specific composition; and like factors well known in the medical arts.

Non-limiting examples of pharmaceutical additives include ingredients to formulate medications to ensure their stability, efficacy, and safety when administered to patients. These carriers are generally inert, non-toxic, and compatible with the active pharmaceutical ingredients (APIs). Below is a list of common pharmaceutically acceptable carriers for oral and intravenous medications:

### Oral Medications

### Solvents and Vehicles

Water
Ethanol
Glycerin
Propylene glycol
Polyethylene glycol (PEG)

### Binders

Microcrystalline cellulose
Starch
Hydroxypropyl methylcellulose (HPMC)
Povidone (polyvinylpyrrolidone, PVP)
Gelatin

### Fillers/Diluents

Lactose
Mannitol
Sorbitol
Dicalcium phosphate
Calcium carbonate

### Disintegrants

Croscarmellose sodium
Sodium starch glycolate
Crospovidone

### Lubricants

Magnesium stearate
Stearic acid
Talc

### Glidants

Colloidal silicon dioxide
Talc

### Coating Agents

Hydroxypropyl methylcellulose (HPMC)
Polyvinyl alcohol (PVA)
Shellac

### Flavoring Agents

Natural and artificial flavors
Sweeteners like sucrose, aspartame, and saccharin

### Coloring Agents

FD&C dyes
Natural colorants like beet juice and caramel

### Intravenous Medications

### Solvents and Vehicles

Water for injection (WFI)
Saline solution (0.9% sodium chloride)
Dextrose solution (5% or 10% dextrose)
Ringer's lactate solution

### Stabilizers

EDTA (ethylenediaminetetraacetic acid)
Citric acid
Ascorbic acid
Sodium metabisulfite

### Buffers

Sodium phosphate
Sodium acetate
Sodium citrate

### Surfactants

Polysorbate 80 (Tween 80)
Polysorbate 20 (Tween 20)
Cremophor EL

### Preservatives

Benzyl alcohol
Phenol
Methylparaben
Propylparaben

### Tonicity Adjusters

Sodium chloride
Dextrose
Mannitol

### Chelating Agents

EDTA
Citric acid

### Antioxidants

Ascorbic acid
Tocopherols (Vitamin E)
Butylated hydroxytoluene (BHT)

These carriers and excipients are chosen based on their compatibility with the active ingredients and the intended route of administration, ensuring that the medication is safe and effective for patient use.

### EXAMPLE

### Example 1—Materials and Methods

### Animal

Nude mice, C57/BL6 mice, and Babl/c mice were purchased from the Laboratory Animal Research Unit (LARU), City University of Hong Kong. All mice and maintained in pathogen-free conditions with a 12 h light/dark cycle. All animal studies were performed according to the guidelines approved by the Animal Ethics Committee of City University of Hong Kong.

### A549 Xenograft Model in Nude Mice

A549 cells (1×10⁶) were injected subcutaneously into 6-8 weeks-old female nude mice. After the diameter of the tumor reached approximately 4 cm, the mice were given vehicle (PEG400/Ethanol/tween80, 1:1:1), 50 mg/kg API orally every day. The size of the tumor and weight of the mice were measured twice a week. At the endpoint, the mice were euthanized, and tumors were harvested for further analysis.

### LLC Xenograft Model in C57/BL6 Mice

LLC cells (1×10⁵) were injected subcutaneously into 6-8 weeks-old female C57/BL6 mice. After the diameter of the tumor reached ˜4 cm, the mice were given vehicle or 50 mg/kg API orally every day. The size of the tumor and weight of the mice were measured twice a week. At the endpoint, the mice were euthanized, and tumors were harvested for further analysis. Total RNA of the tumor was extracted by RNAiso Plus (Takara, T9108) according to the manufacturer's protocol. Expression levels of relative genes were analyzed by RT-qPCR.

### B16F10 Xenograft Model in C57/BL6 Mice

B16F10 cells (1×10⁵) were injected subcutaneously into 6-8 weeks-old female C57/BL6 mice. After the diameter of the tumor reached ˜5 cm, the mice were given vehicle or 50 mg/kg API orally every day. The size of the tumor and weight of the mice were measured twice a week. At the endpoint, the mice were euthanized, and tumors were harvested for further analysis. Total RNA of the tumor was extracted by RNAiso Plus (Takara, T9108) according to the manufacturer's protocol. Expression levels of relative genes were analyzed by RT-qPCR.

### a Combination of AP1 and PD-1 in LLC Xenograft Model

LLC cells (1×10⁵) were injected subcutaneously into 6-8 weeks-old female C57/BL6 mice. After the diameter of the tumor reached ˜5 cm, the mice were given vehicle (daily) plus Isotype (200 μg/mouse, twice a week), 50 mg/kg AP1 (daily), PD1 (200 μg/mouse, twice a week) plus 50 mg/kg AP1 (daily). The size of the tumor and weight of the mice were measured twice a week. At the endpoint, the mice were euthanized, and tumors were harvested for further analysis.

### A549 Metastasis Mouse Model in NSG Mice

A549-luc cells (1×10⁵) were injected via tail vein into 6-8 weeks-old female NSG mice. On the next day, the mice were randomly divided into two groups (n=4 per group) and treated with either buffer or AP1 (30 mg/kg, oral, daily) for 19 days. During the course of the experiment, the luminescence and body weight of the mice were measured every 3-4 days. At the end of the experiment, the lungs in each group were collected and imaged, and the luminescent signals were quantified.

### Example 2

### Ability of Synthetic 1,3,5-Triazine Derivatives to Induce Cell Vacuolization

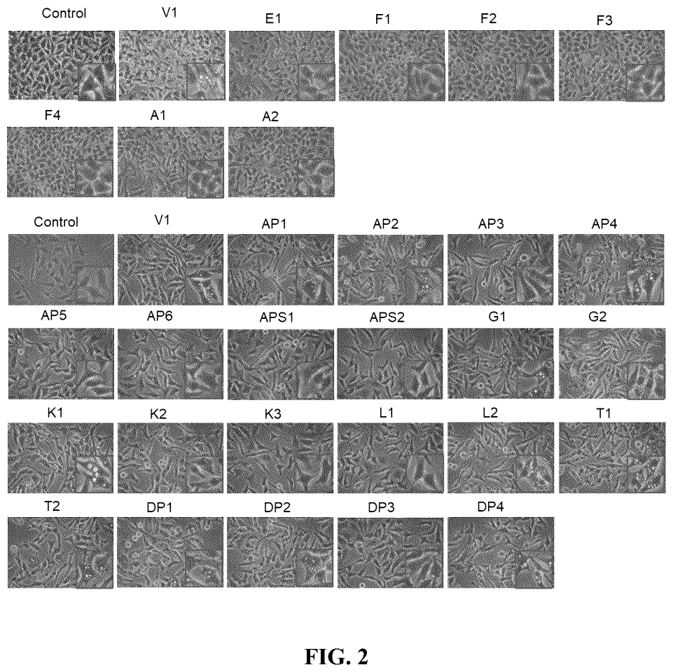

The abilities of these 27 novel triazine compounds to modulate cell morphology or viability are then screened. As shown in FIG. 2, after treating HeLa cells with 1 μM of these novel triazine compounds, many of them, including AP1, AP2, AP4, G₁, G₂, K₁, K₂, L2, T1, T2, DP1, DP2, and DP4, induced obvious vacuoles in cells. However, none of these compounds affects cell viability at this concentration.

### Example 3

### Assessment of Triazine Derivatives on Autophagy Regulation and Potency

Since dysregulated autophagy or endosomal trafficking may lead to vacuole formation, the potential regulation of autophagy by these triazine derivatives was then assessed.

During autophagy, LC3 is processed and converted from its cytosolic form (LC3-I) to a lipidated form (LC3-II) that associates with autophagosome membranes. The presence of LC3-II is indicative of autophagosome formation, making it a useful marker for studying autophagy. p62 serves as a cargo receptor in autophagy. It binds to ubiquitinated proteins and delivers them to autophagosomes for degradation. During autophagy, p62 itself is also degraded.

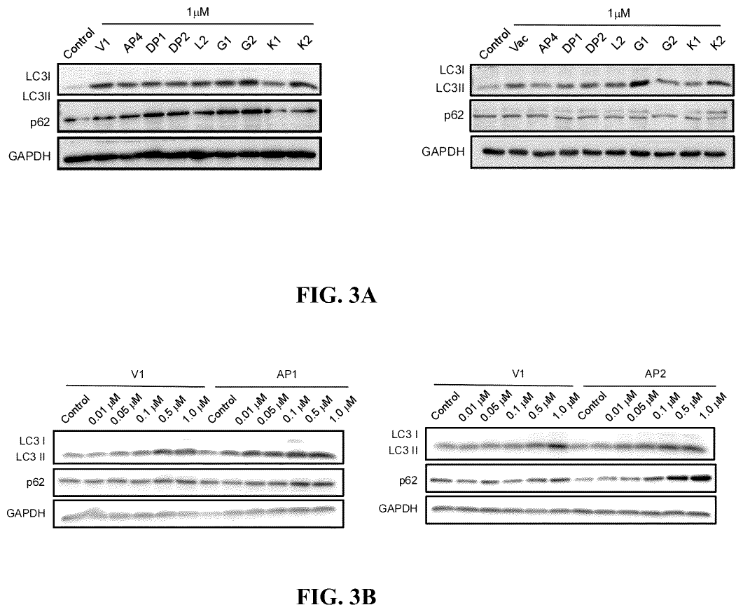

Referring to FIG. 3A, these novel triazine compounds (such as AP4, DP1, DP2, L2, G1, G2, K1 and K2), similar to V1 (a known autophagy inhibitor), also inhibited autophagy, as evidenced by an accumulation of LC3-II and p62 in cells treated with these compounds. The accumulation of LC3-II and p62 indicated impaired autophagic flux or inhibition of autophagy.

Furthermore, FIG. 3B showed that API and AP2 caused a dose-dependent accumulation of LC3-II and p62, and at a concentration of 1 μM, their ability to inhibit autophagy was comparable to that of V1. Of note, compound API or AP2 was able to inhibit autophagy even at a concentration as low as 0.05 μM. These results suggested that API and AP2 were more potent than other triazine compounds in inhibiting autophagy.

### Example 4

### Modulation of Endosomal Trafficking by Triazine Derivatives

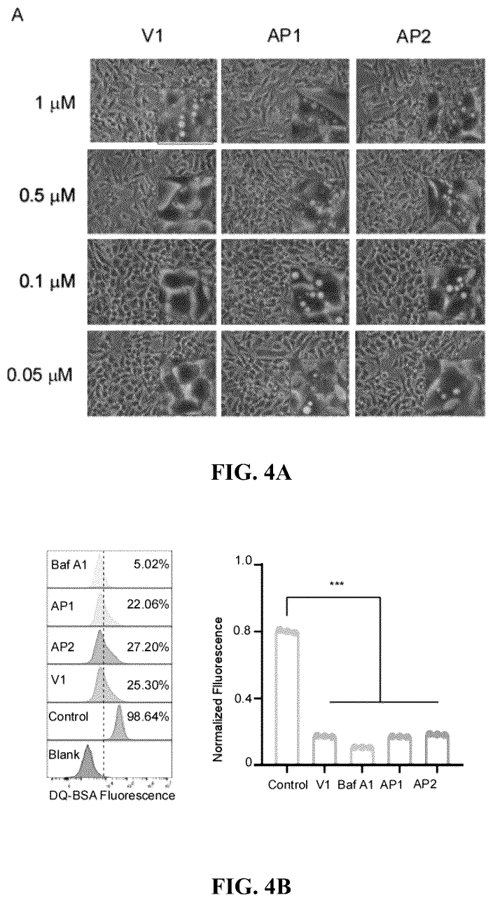

The ability of API and AP2 to modulate endosomal trafficking was determined. Referring to FIG. 4A, AP1 or AP2 induced vacuoles at a concentration as low as 0.05 μM, whereas V1 only induced large vacuoles at 1 μM.

DQ-BSA is a self-quenched fluorescent derivative of bovine serum albumin (BSA), specifically labeled with a fluorophore. In its intact form, the fluorescence of DQ-BSA is quenched, meaning that it does not emit a fluorescent signal. However, upon proteolytic cleavage by intracellular enzymes, the quenching is relieved, resulting in a fluorescent signal that can be detected. This property makes DQ-BSA a valuable tool for monitoring endosomal and lysosomal degradation activities within cells. It is particularly useful in studying cellular processes such as autophagy, protein trafficking, and the general proteolytic activity of intracellular compartments. The resulting fluorescence provides real-time insights into the dynamic processes occurring within endosomes and lysosomes, allowing researchers to better understand the mechanisms underlying these critical cellular functions. FIG. 4B showed that API or AP2, similar to V1 or bafilomycin A1 (Baf A1), significantly inhibited the degradation of DQ-BSA, indicating that API or AP2 also inhibits endosomal trafficking.

In addition, the ability of API to inhibit autophagy was assessed using the autophagic flux assay. In this assay, GFP-RFP-LC3 expressing HeLa cells were used to monitor autophagic activity by exploiting the different pH sensitivities of the GFP and RFP fluorescent proteins, which were fused to LC3, a key autophagy marker. GFP fluorescence was quenched in acidic environments, such as lysosomes, while RFP remained stable. During autophagy, LC3 transitioned from autophagosomes (neutral pH, both GFP and RFP positive) to autolysosomes (acidic pH, only RFP positive). By monitoring the changes in fluorescence, the effects of AP1, BafA1, or a combination of both on the progression of autophagosomes (GFP⁺RFP⁺LC3 puncta) to autolysosomes (RFP⁺ LC3 puncta) were assessed.

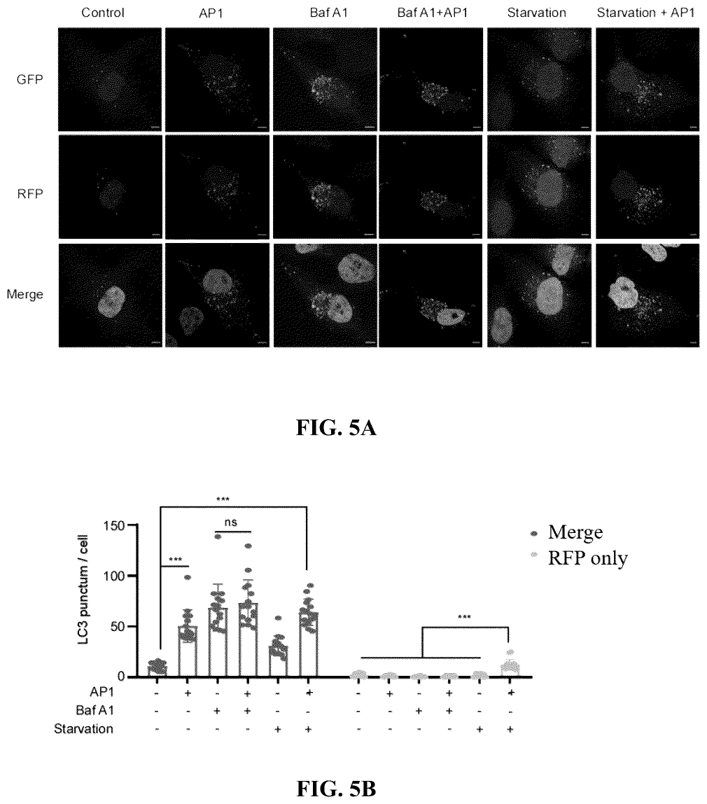

Referring to FIGS. 5A-5B, Hela cells expressing RFP-GFP-LC3 were treated with 1 μM AP1, 100 nm Baf A1, or amino acid starvation alone, or with a combination of these for 24 h. The cells treated with API or Baf A1 alone accumulated yellow, fluorescent LC3 puncta, indicating the accumulation of autophagosomes. When cells were co-treated with API and Baf A1, there was no further increase in LC3 yellow fluorescent puncta. Amino acid starvation (AAS), as a positive autophagy inducer control, induced both yellow and red-only LC3 puncta, and a combination of AAS and API significantly increased the number of LC3 yellow puncta. These results indicated that API, similar to Baf A1, inhibited autophagosome-lysosome fusion. In conclusion, two novel 1,3,5-triazine compounds, AP1 and AP2, were potent inhibitors of endosomal trafficking and autophagy.

### Example 5

### In Vitro Antitumor Activity of Triazine Derivatives

In this example, the antitumor activities of API and AP2 in vitro were assessed. Various cancer cell lines were treated with either API or AP2, and the viability of the cells was tested. The cancer cells included Hela cells (the human cervical cancer cell line), A375 cells (the human melanoma cell line), A549 (the human lung adenocarcinoma cell line), and LLC (the Lewis mouse lung carcinoma cell line).

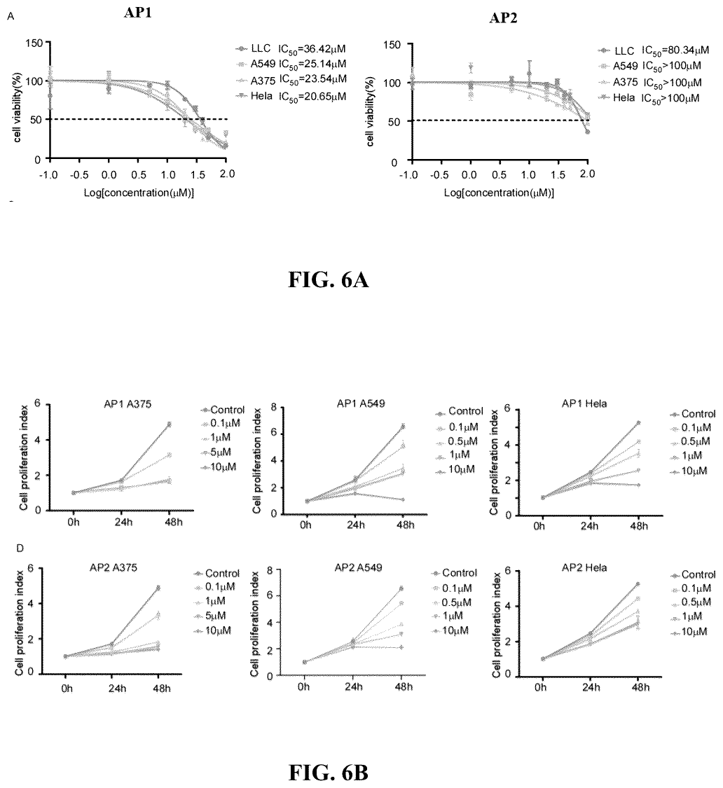

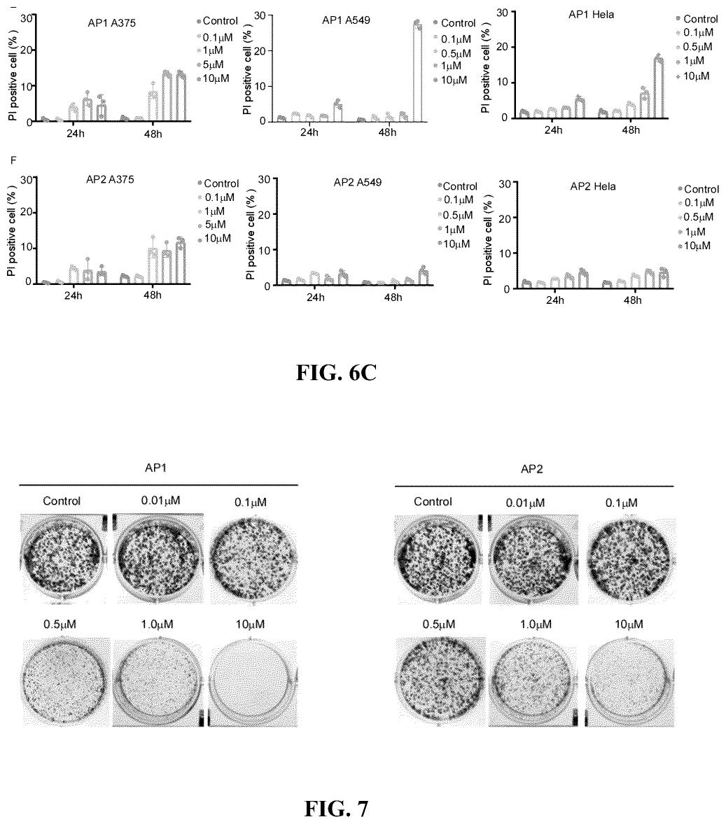

Referring to FIG. 6A, the MTT results showed that API exhibited IC50 values of 20.65 μM, 23.54 μM, 25.15 μM, and 36.42 μM in the HeLa, A375, A549, and LLC cells, respectively. In contrast, AP2 showed significantly lower toxicity, with IC50 values of >100 μM for most of these cell lines. This suggested that AP1 may have greater potency in killing cancer cells.

Moreover, the impact of API or AP2 on cell proliferation was also evaluated, as this process is closely linked to endosomal trafficking. The HeLa, A375 or A549 cells were treated with 0, 0.1, 0.5, 5, or 10 μM AP1 or AP2 for 24 h or 48 h. FIG. 6B demonstrated that AP1 or AP2 inhibited the proliferation of A375, A549, and HeLa cells at concentrations>1 μM. In addition, FIG. 6C showed that AP1, but not AP2, caused obvious cell death in A549 and HeLa cells as evidenced by the propidium iodide (PI) staining results, which again suggested that API might have better antitumor activity.

In subsequent experiments, A549 cells were utilized to investigate the bioactivity of API and AP2. First, a colony formation assay was conducted to assess the ability of API or AP2 to inhibit the clonogenic potential of A549 cells. A549 cells were seeded into 12-well plates (1,000 cells/well) and cultured for 24 h, after which they were treated with 0.01, 0.1, 0.5, 1, or 10 μM, and incubated for 7 days. Cells were then stained with crystal violet. FIG. 7 illustrated that API effectively suppressed colony formation of A549 cells at a concentration as low as 0.5 μM, whereas AP2 required a higher concentration of 10 μM to achieve similar inhibition.

### Example 6

### Effect of Triazine Derivatives on Cell Migration

Given the critical role of endosomal trafficking in cell migration, the transwell assay and wound healing assay were conducted to evaluate the impact of AP1 or AP2 on cell migration.

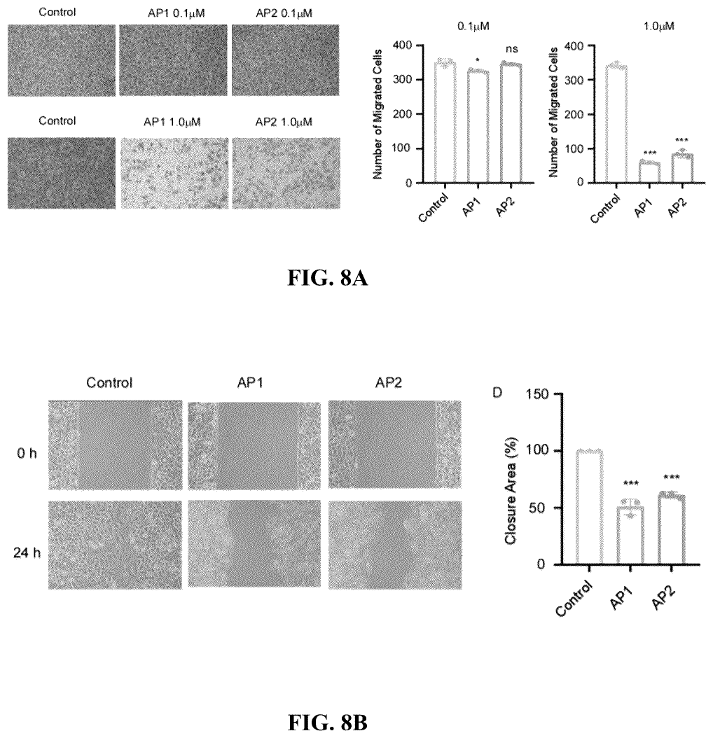

In the transwell assay, 300,000 A549 cells were seeded into the upper well of a transwell chamber. After 24 h, the cells were treated with 0.1 μM or 1 μM of either API or AP2. In the wound healing assay, 400,000 A549 cells were seeded into a 6-well plate with a culture insert placed in the middle of the well. After 24 h, the insert was removed to create a clear zone. Subsequently, the cells were treated with 1 μM AP1 or AP2 for 24 h. Referring to FIGS. 8A-8B, both API and AP2 inhibited cell migration and wound healing of A549 cells, with API showing slightly greater efficiency than AP2.

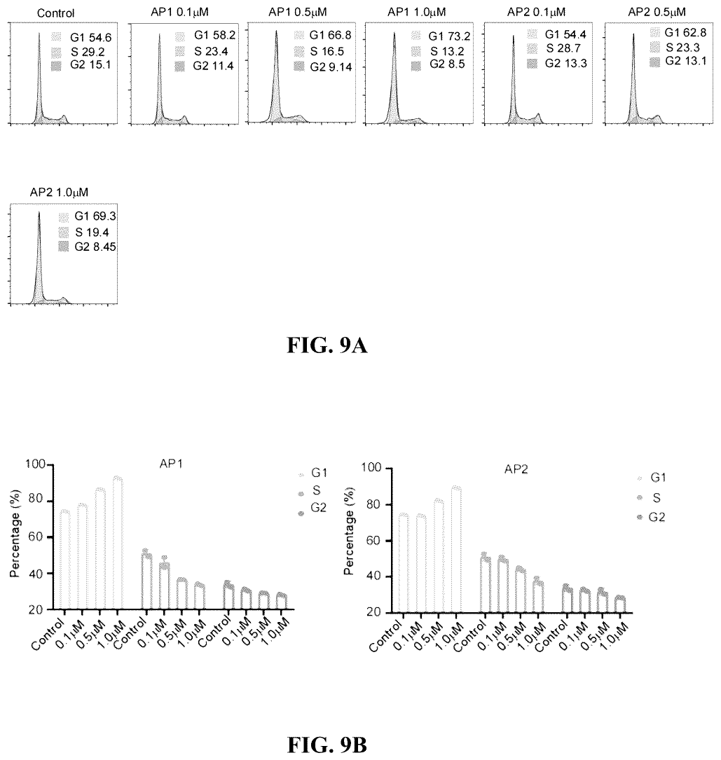

In light of API or AP2 inhibiting cell proliferation without apparent toxicity at low concentrations, a flow cytometric assay was conducted to assess their impact on the cell cycle of A549 cells. In FIGS. 9A-9B, A549 cells treated with AP1 or AP2 exhibited dose-dependent arrest in the G0/G1 phase. The effect of API was more significant compared to AP2, as there was an obvious arrest at a concentration of 0.5 μM and a maximum increase of 19% at the highest concentration of 1 μM.

### Example 7

### Effect of Triazine Derivatives on Apoptosis and Necrosis

Due to AP1 causing cell death at high concentrations, Annexin-V-FITC and PI dual staining was conducted to verify the capability of AP1 or AP2 to induce cell death. FITC (fluorescein isothiocyanate)-labeled Annexin-V binds to phosphatidylserine exposed on the outer membrane of apoptotic cells, marking the early stages of apoptosis. PI (propidium iodide) is a nuclear dye that penetrates cells with compromised membranes, often used alongside Annexin-V-FITC to distinguish between apoptosis and necrosis. Cells stained only with Annexin-V-FITC indicate apoptosis, while those that are double-positive for both Annexin-V and PI are undergoing necrosis.

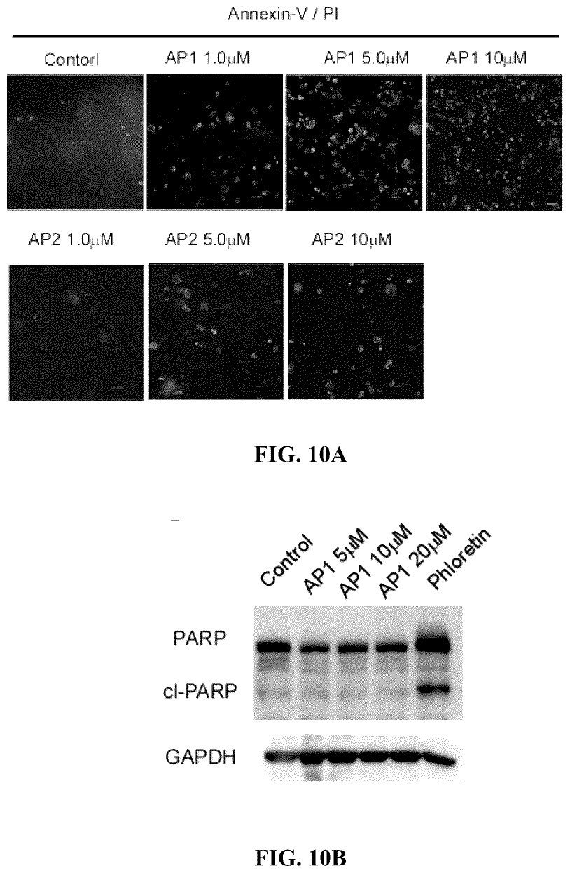

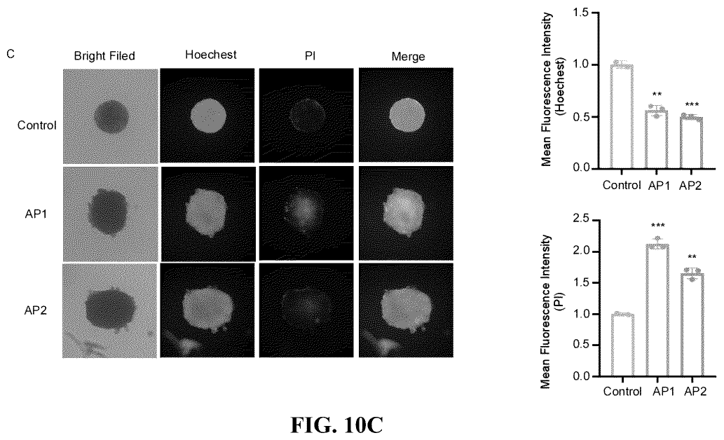

The A549 cells were seeded at the same density and treated with 0, 1.0, 5.0, or 10 μM AP1 or AP2, followed by co-staining with Annexin-V and PI. Referring to FIG. 10A, AP1 induced significant necrosis at a concentration of 10 μM, whereas AP2 had minimal impact on cell death. These findings were consistent with the results shown in FIG. 6C. For AP1 group, there was no significant change in these markers even at a concentration of 20 μM (FIG. 10B), suggesting that the cell death induced by AP1 operated independently of the canonical apoptotic pathway.

Next, the involvement of the apoptosis pathway in cell death was investigated by examining the level of cleaved PARP in cells treated with or without AP1. The A549 cells were treated with 5, 10, or 20 μM AP1 for 4 h or with 300 μM phloretin for 24 h. The positive control used phloretin, which could significantly increase the levels of PARP and cleaved PARP, indicating the initiation of apoptosis. The levels of PARP and Cl-PARP were then analysed by Western Blot.

Cancer stem cells (CSCs) are a subpopulation of cells that have been found in various solid tumors, such as breast, prostate, and lung cancers. CSCs are important for tumor initiation, recurrence, metastasis, and drug resistance. In vitro, tumor spheres are solid, spherical structures formed by a single cell or a small population of cells that mimic the population of stem cells residing in tumors. Tumor spheres have been universally used in drug screening and development studies¹⁻³.

Accordingly, the present invention constructed an A549 tumor sphere formation assay to evaluate the antitumor activity of triazine derivatives. Hoechst dye binds to DNA, marking the nucleus. Changes in nuclear structure or DNA degradation during cell death or apoptosis can lead to reduced Hoechst staining intensity or abnormal nuclear morphology. When Hoechst staining decreases and PI staining increases, it may indicate cell death, particularly necrosis, with potential effects on nuclear structure and DNA integrity. Referring to FIG. 10C, both AP1 and AP2 significantly inhibited cell proliferation and induced cell death in the tumor sphere, as indicated by the decreased fluorescence of Hoechst and the dramatically increased fluorescence of PI after treatment with 20 μM of either compound. The size of the tumor sphere in the compound-treated group was larger than that in the control group. This was because of the dissociation of cells in the tumor sphere, which resulted in a loose, less compact structure when compared with the control.

Taken together, the above results indicated that triazine derivatives, especially AP1 and AP2, were potential antitumor agents.

### Example 8

### In Vivo Antitumor Activity Study of Triazine Derivatives

Given the promising in vitro antitumor activity exhibited by AP1 and AP2, various animal models were established to assess their in vivo efficacy against tumors.

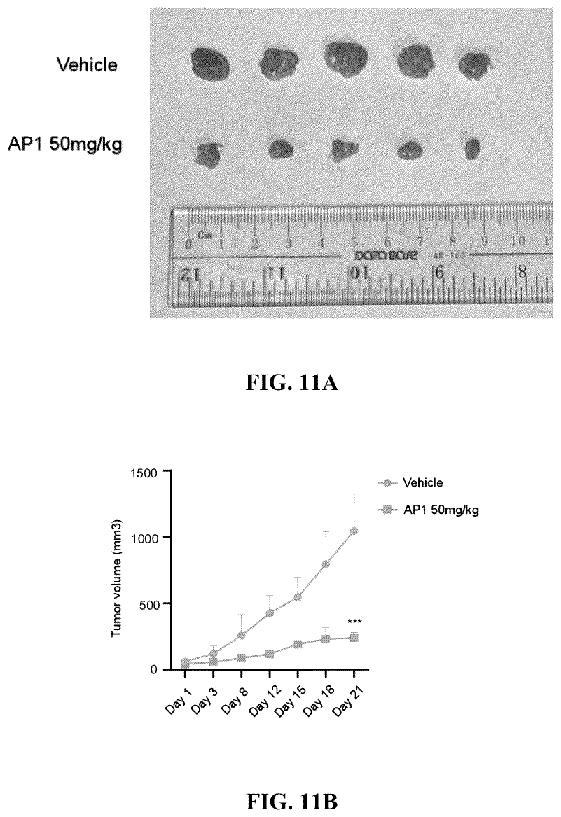

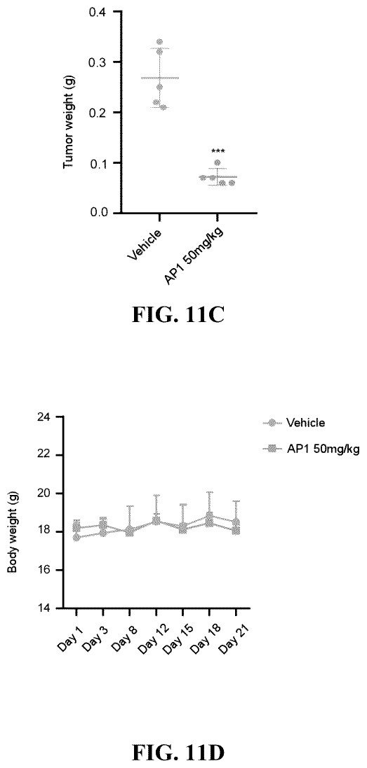

Initially, a xenograft tumor model was established by subcutaneously transplanting A549 cells into nude mice. After the size of the tumor reached approximately 5 mm in diameter (FIG. 11A), the mice were treated with AP1 (50 mg/kg) daily via oral administration for 21 days. It could be seen that AP1 significantly inhibited the tumor volume and tumor weight (FIG. 11B-11C). However, the administration of AP1 did not cause a noticeable loss in the body weight of the mice, which suggested its low toxicity (FIG. 11D). These results suggested that AP1 had in vivo antitumor activity.

### Example 9

### Effect of Triazine Derivatives on Tumor Microenvironment Polarization

Macrophages are dynamic cells that can be polarized into either M1 or M2 type, and they play critical roles in regulating innate immunity. The polarization of M1 requires the coordination of Th1 cytokines such as colony-stimulating factor (e.g., GM-CSF), tumor necrosis factor α (TNF-α), and interferon-γ (IFN-γ), as well as lipopolysaccharide (LPS). M1 macrophages express special markers including CD80, CD86, and TLR, and they secrete pro-inflammatory cytokines such as interleukin-1β (IL-1β), IL-6, IL-12, IL-23, and TNF-α. In contrast, M2 macrophages are polarized by Th2 cytokines such as IL-4 and IL-13, and they produce anti-inflammatory cytokines such as IL-10 and transforming growth factor beta (TGF-β). Tumor-associated macrophages (TAMs) are suggested to be important for tumor development. In addition, cytokines secreted by TAMs are regulators of the immune response.

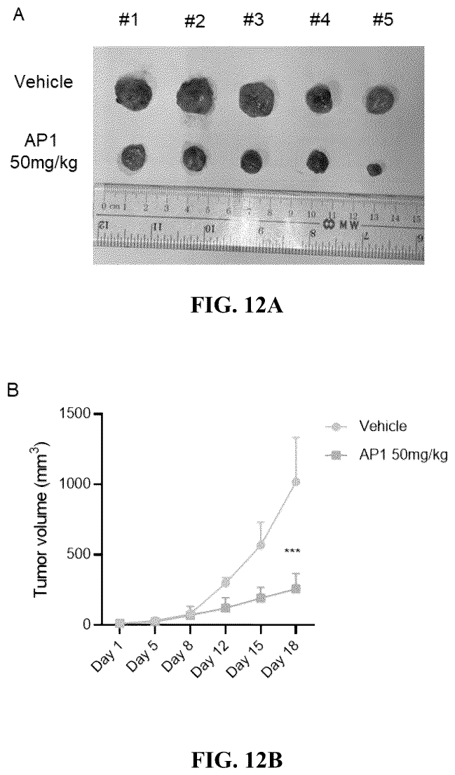

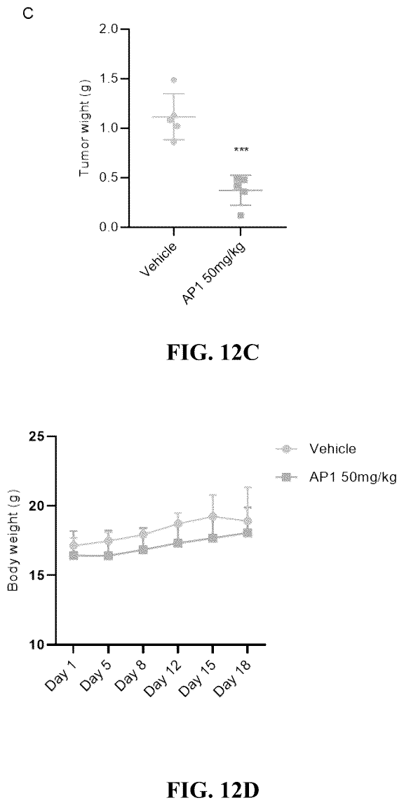

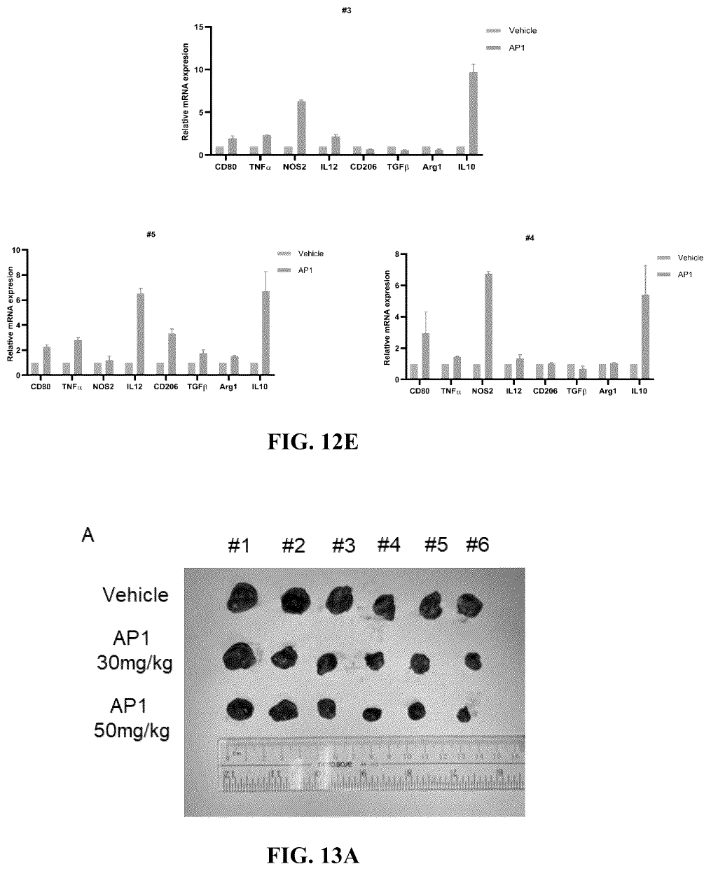

Considering the relationship between endosomal trafficking and the tumor microenvironment (TME), xenograft tumor mouse models were established in normal mice with intact immune systems. Lewis lung carcinoma (LLC) cells were subcutaneously transplanted into C57/BL6 mice. After the diameter of the tumor reached approximately 4 mm in diameter (FIG. 12A), the mice were treated with AP1 (at 50 mg/kg/mouse) orally once a day for 18 days. FIGS. 12B-12C showed that AP1 greatly suppressed tumor growth with a decrease in both tumor weight and tumor size. The administration of AP1 did not cause a noticeable loss in the body weight of the mice, which suggested its low toxicity (FIG. 12D).

To assess the effect of AP1 on the TME, the level of macrophages and related cytokines secreted by primary tumors was evaluated. RT-qPCR was conducted to examine the gene expression levels of the MI marker CD80 and related cytokines, including TNF-α, IL-12, and NOS2, in primary tumors isolated from mice treated with or without AP1. Likewise, the gene expression levels of the M2 marker, CD206, and its cytokines IL-10, TGF-β, and Arg1, were also examined.

Referring to FIG. 12E, AP1 induced a higher level of the M1 marker CD80 and its associated cytokines (e.g., TNF-α, IL-12, NOS2). Of note, in one primary isolated from AP1-treated mouse, AP1 slightly increased the level of M2 marker CD206, and its cytokine TGFβ and Arg1. This indicated a positive regulation of immune function as the M1 subtype usually enhances phagocytosis of cancer cells and antitumor inflammatory reactions. However, IL-10 was also found to be significantly increased, which could be attributed to hyperactivation of the immune response.

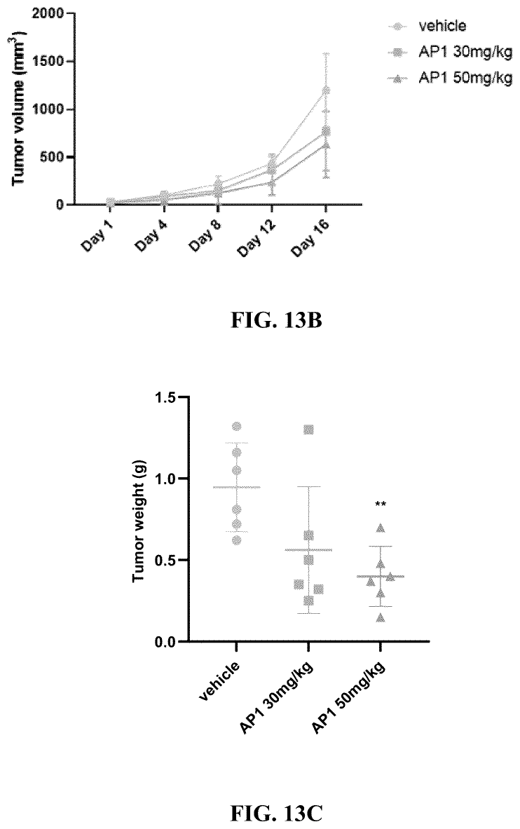

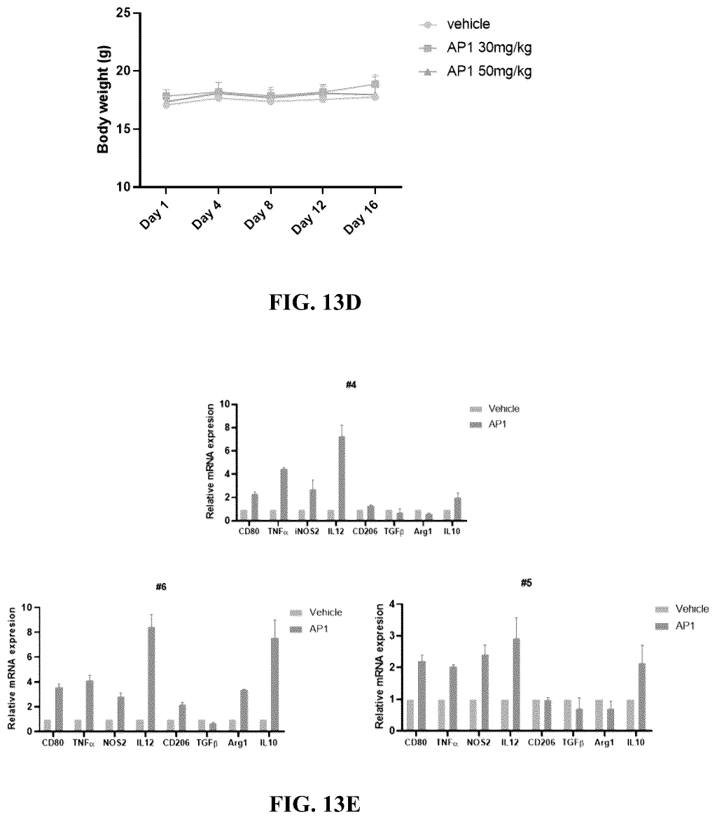

To validate AP1's impact on the TME, an orthotopic model was constructed by subcutaneously injecting B16F10 cells (mouse melanoma cells) into C57/BL6 mice. After the tumor reached approximately 5 mm in diameter (FIG. 13A), the mice were treated with AP1 (50 mg/kg/mouse or 30 mg/kg/mouse) orally once a day for 16 days. FIGS. 13B-13C showed that AP1 at both doses greatly suppressed tumor growth and decreased tumor weight. AP1 also induced a higher level of M1 macrophages and its associated cytokines in primary tumors isolated from the AP1-treated mice, and the M2 cytokine IL-10 also increased. These findings were highly consistent with the LLC xenograft model results (FIGS. 12A-12E).

Given that AP1 influenced the levels of M1 macrophages and cytokine secretion in primary tumors, this investigation extended to exploring its potential in combination with the immune checkpoint inhibitor, anti-PD1 antibody.

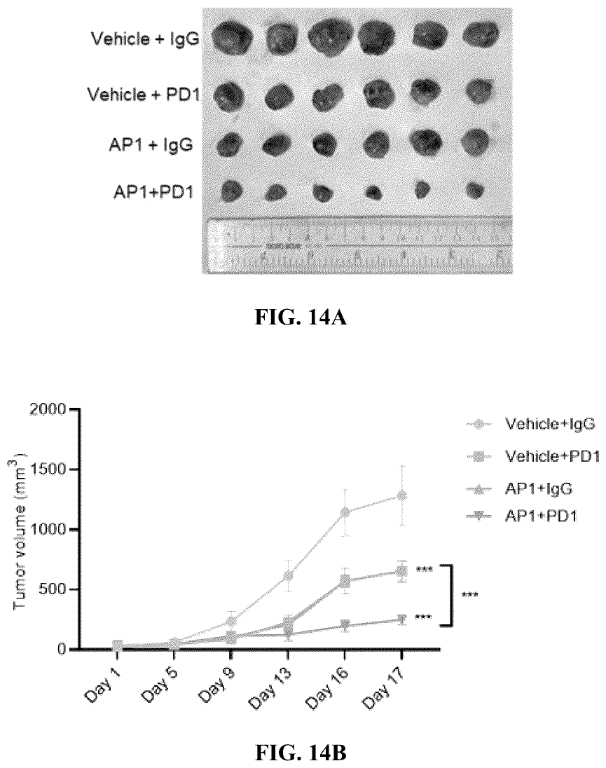

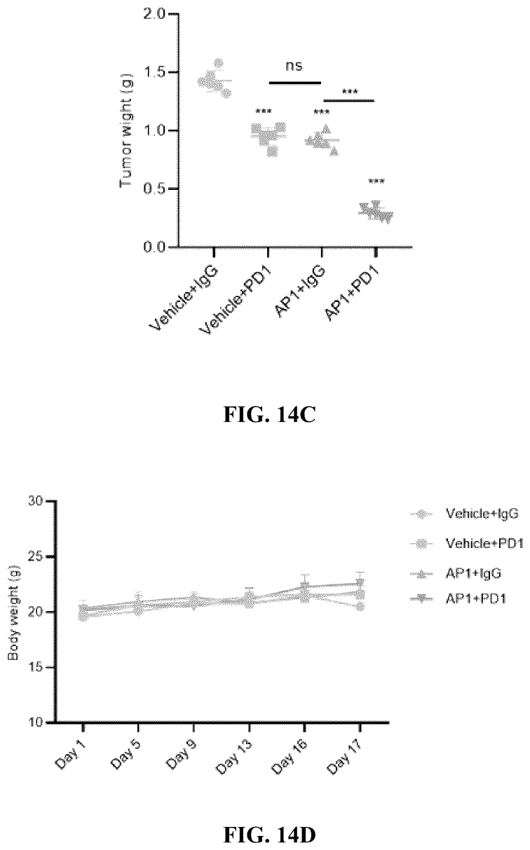

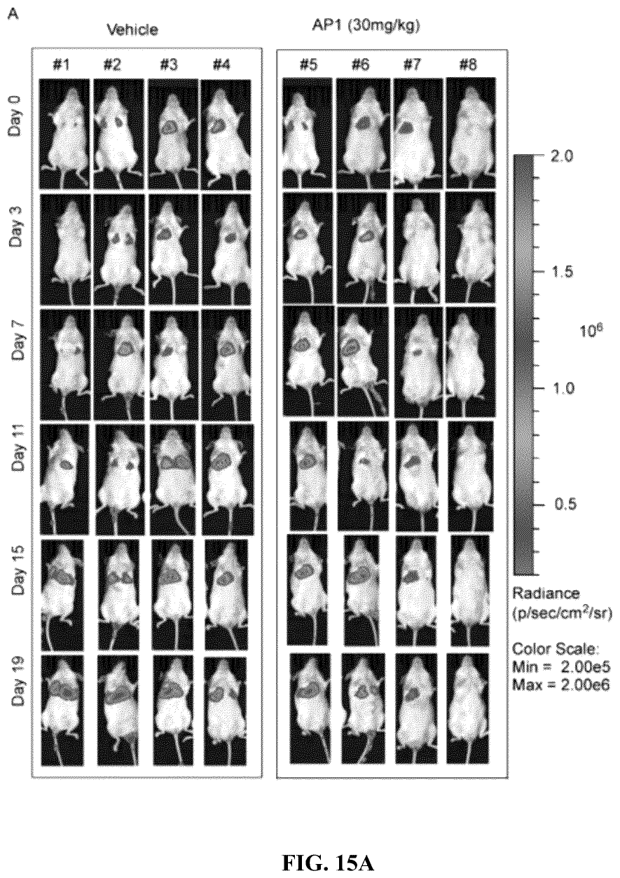

LLC cells were subcutaneously transplanted into C57/BL6 mice. After the diameter of the tumor reached approximately 5 mm in diameter (FIG. 14A), the mice were divided into 4 groups randomly: (1) Vehicle+IgG, (2) PD1+Vehicle, (3) AP1+IgG, or (4) AP1+PD1. AP1 was administered orally at 50 mg/kg/mouse once daily, while IgG or PD1 was administered via intraperitoneal injection at 200 μg/mouse twice weekly.

Turning to FIGS. 14B-14C, AP1 significantly suppressed tumor growth, manifested by a decrease in tumor weight and size, and the PD1 antibody also showed a comparable activity. Combining AP1 with the PD1 antibody resulted in a further reduction in both tumor size and weight without additional toxicity to the mice compared to either treatment alone, indicating a synergistic effect between the two drugs. These findings further suggested that AP1 might have augmented the antitumor immune response, underscoring its potential as a complementary agent in combination with other immunotherapy treatments.

### Example 10

### Anti-Metastasis Activity of Triazine Derivatives

After AP1 effectively suppressed cancer cell migration, the evaluation of its anti-metastatic activity was conducted in an experimental mouse model. The tail vein injection model is commonly used to study the late stages (extravasation and metastatic colonization) of metastasis. This includes the tail vein injection of A549-Luc2 cells expressing luciferase (Fluc−-expressing A549 cells) into nude mice.

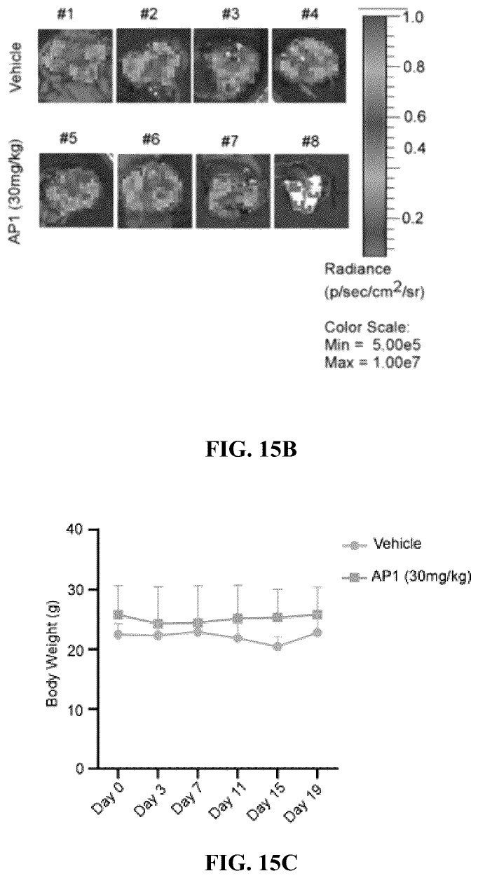

One day after injection, the mice were randomly divided into two groups (n=4 per group) and treated with either buffer or AP1 (30 mg/kg, oral, daily) for 19 days. During the course of the experiment, the mice were imaged every 3-4 days, and the luminescent signals were quantified. FIG. 15A showed that in the AP1-treated mice, far weaker luminescent signals were spread over a smaller area during the course of drug treatment when compared to the controls. After 19 days, the mice were euthanized and the lungs were isolated and subjected to fluorescence imaging. The lungs isolated from the AP1-treated mice showed much weaker signals when compared to the control (FIG. 15B). The administration of AP1 did not cause a noticeable loss in the body weight of the mice.

As anticipated, AP1 treatment had minimal effects on the body weights of the mice. These findings suggested that AP1 also inhibited the metastasis of malignant tumors in vivo.

In summary, the present invention provides a series of novel 1,3,5-triazine derivatives as endosomal trafficking inhibitors. These triazine derivatives, especially AP1 and AP2, can act as potential antitumor agents. Furthermore, these triazine derivatives exhibit synergistic effects when combined with the PD1 antibody.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of +10%, +5%, +1%, or +0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within +10%, +5%, +1%, or +0.5% of the average of the values.

The term “subject” in particular refers to an animal or human, in particular a mammal and most preferably human. The expression “effective amount” generally denotes an amount sufficient to produce therapeutically desirable results, wherein the exact nature of the result varies depending on the specific condition which is treated.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

### REFERENCES: THE DISCLOSURES OF THE FOLLOWING REFERENCES ARE INCORPORATED BY REFERENCE

1. Bahmad, H. F., Cheaito, K., Chalhoub, R. M., Hadadeh, O., Monzer, A., Ballout, F., El-Hajj, A., Mukherji, D., Liu, Y. N., Daoud, G., and Abou-Kheir, W. (2018). Sphere-Formation Assay: Three-Dimensional in vitro Culturing of Prostate Cancer Stem/Progenitor Sphere-Forming Cells. Front Oncol 8, 347. 10.3389/fonc.2018.00347.
2. Singh, S. K., Abbas, S., Saxena, A. K., Tiwari, S., Sharma. L. K., and Tiwari. M. (2020). Critical role of three-dimensional tumorsphere size on experimental outcome. Biotechniques 69. 333-338. 10.2144/btn-2020-0081.
3. Johnson. S., Chen. H., and Lo. P. K. (2013). In vitro Tumorsphere Formation Assays. Bio Protoc 3. 10.21769/bioprotoc.325.

1. A method for treating a malignant cancer in a subject in need thereof, comprising administering a therapeutically effective amount of a pharmaceutical composition to the subject, wherein the pharmaceutical composition comprises a 1,3,5-triazine derivative and a pharmaceutically acceptable carrier.

2. The method of claim 1, wherein the malignant cancer comprises a breast cancer, lung cancer, colorectal cancer, prostate cancer, pancreatic cancer, liver cancer, esophageal cancer, gastric cancer, ovarian cancer, cervical cancer, endometrial cancer, renal cancer, bladder cancer, thyroid cancer, melanoma, non-Hodgkin lymphoma, Hodgkin lymphoma, multiple myeloma, leukemia, glioblastoma, astrocytoma, medulloblastoma, meningioma, sarcoma, bone cancer, head and neck cancer, testicular cancer, oral cancer, anal cancer, mesothelioma, neuroblastoma, retinoblastoma, and cholangiocarcinoma.

3. The method of claim 2, wherein the malignant cancer is selected from melanoma or lung carcinoma.

4. The method of claim 1, wherein the 1,3,5-triazine derivative comprises the following structural formulas:

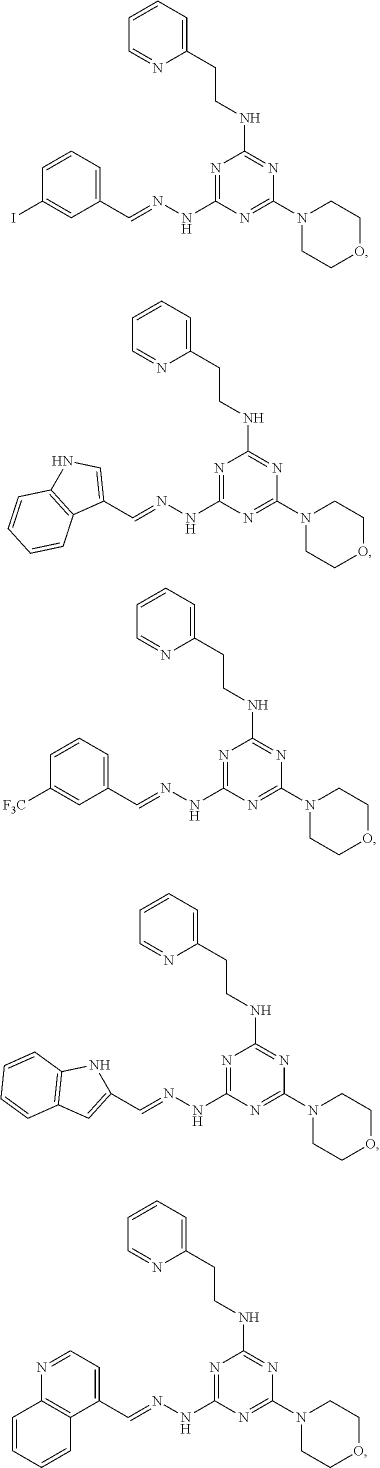 [SMILES: Ic1cccc(/C=N/Nc2nc(NCCc3ccccn3)nc(N3CCOCC3)n2)c1 ; C(=N\Nc1nc(NCCc2ccccn2)nc(N2CCOCC2)n1)/c1c[nH]c2ccccc12 ; FC(F)(F)c1cccc(/C=N/Nc2nc(NCCc3ccccn3)nc(N3CCOCC3)n2)c1 ; C(=N\Nc1nc(NCCc2ccccn2)nc(N2CCOCC2)n1)/c1cc2ccccc2[nH]1 ; C(=N\Nc1nc(NCCc2ccccn2)nc(N2CCOCC2)n1)/c1ccnc2ccccc12]

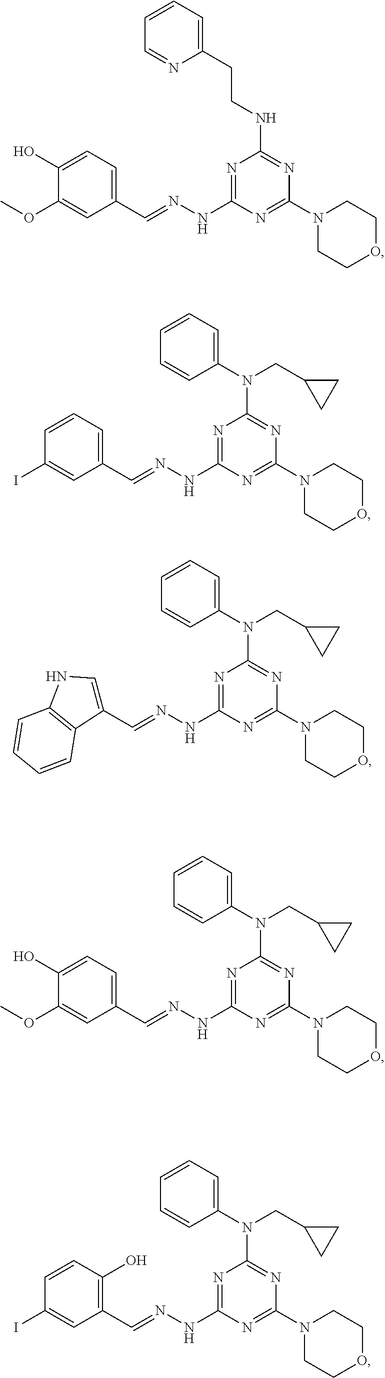 [SMILES: COc1cc(/C=N/Nc2nc(NCCc3ccccn3)nc(N3CCOCC3)n2)ccc1O ; Ic1cccc(/C=N/Nc2nc(N3CCOCC3)nc(N(CC3CC3)c3ccccc3)n2)c1 ; C(=N\Nc1nc(N2CCOCC2)nc(N(CC2CC2)c2ccccc2)n1)/c1c[nH]c2ccccc12 ; COc1cc(/C=N/Nc2nc(N3CCOCC3)nc(N(CC3CC3)c3ccccc3)n2)ccc1O ; Oc1ccc(I)cc1/C=N/Nc1nc(N2CCOCC2)nc(N(CC2CC2)c2ccccc2)n1]

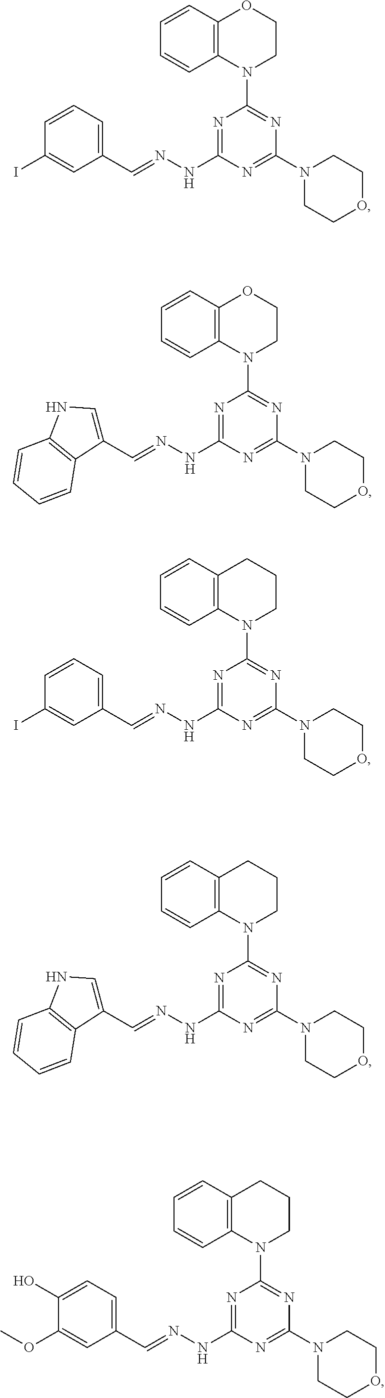 [SMILES: Ic1cccc(/C=N/Nc2nc(N3CCOCC3)nc(N3CCOc4ccccc43)n2)c1 ; C(=N\Nc1nc(N2CCOCC2)nc(N2CCOc3ccccc32)n1)/c1c[nH]c2ccccc12 ; Ic1cccc(/C=N/Nc2nc(N3CCOCC3)nc(N3CCCc4ccccc43)n2)c1 ; C(=N\Nc1nc(N2CCOCC2)nc(N2CCCc3ccccc32)n1)/c1c[nH]c2ccccc12 ; COc1cc(/C=N/Nc2nc(N3CCOCC3)nc(N3CCCc4ccccc43)n2)ccc1O]

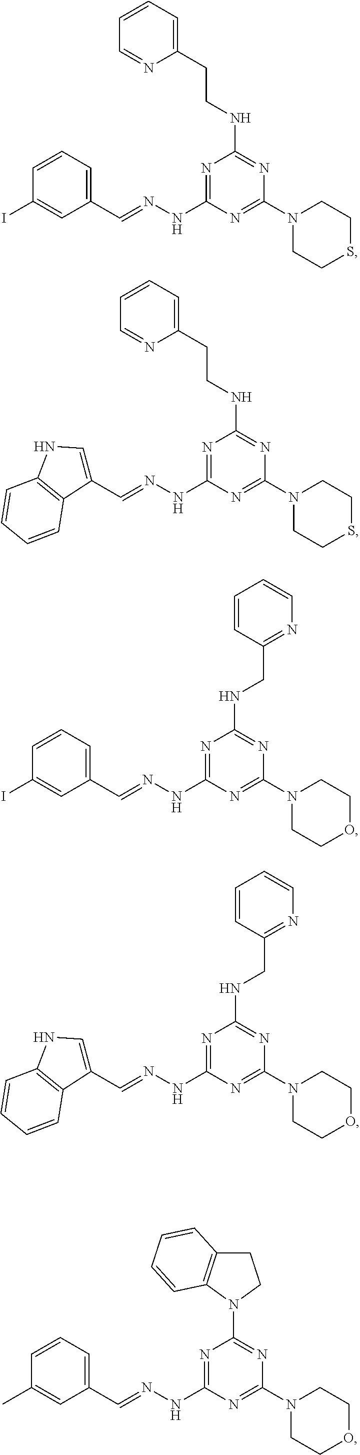 [SMILES: Ic1cccc(/C=N/Nc2nc(NCCc3ccccn3)nc(N3CCSCC3)n2)c1 ; C(=N\Nc1nc(NCCc2ccccn2)nc(N2CCSCC2)n1)/c1c[nH]c2ccccc12 ; Ic1cccc(/C=N/Nc2nc(NCc3ccccn3)nc(N3CCOCC3)n2)c1 ; C(=N\Nc1nc(NCc2ccccn2)nc(N2CCOCC2)n1)/c1c[nH]c2ccccc12 ; Cc1cccc(/C=N/Nc2nc(N3CCOCC3)nc(N3CCc4ccccc43)n2)c1]

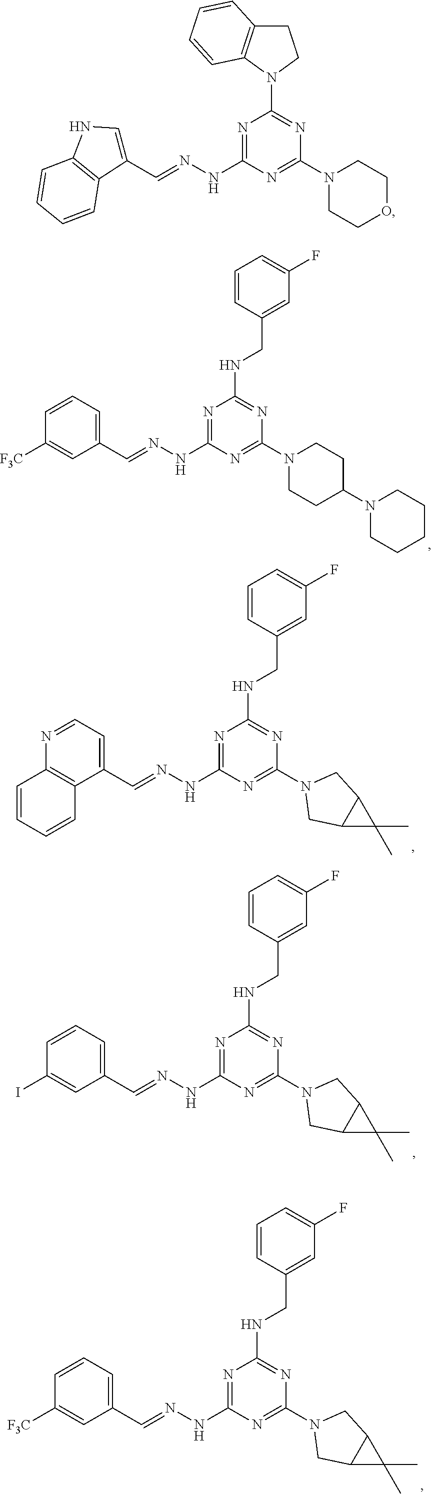

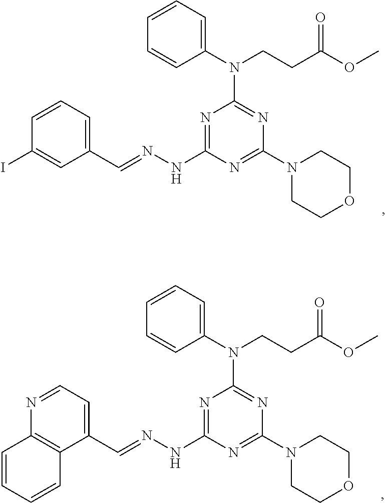 [SMILES: COC(=O)CCN(c1ccccc1)c1nc(N/N=C/c2cccc(I)c2)nc(N2CCOCC2)n1 ; COC(=O)CCN(c1ccccc1)c1nc(N/N=C/c2ccnc3ccccc23)nc(N2CCOCC2)n1]

5. The method of claim 4, wherein the 1,3,5-triazine derivative induces macrophages to polarize into M1 macrophages.

6. The method of claim 1, wherein the pharmaceutically acceptable carrier is selected from the group consisting of diluents, excipients, and stabilizers.

7. The method of claim 4, wherein the pharmaceutical composition inhibits endosomal trafficking of the malignant cancer cells, resulting in induction of necrosis in the malignant cancer cells.

8. The method of claim 4, wherein the pharmaceutical composition inhibits autophagy in the malignant cancer cells and the proliferation of the malignant cancer cells.

9. The method of claim 1, wherein the pharmaceutical composition inhibits colony formation, migration, and tumor sphere formation in the malignant cancer cells.

10. The method of claim 1, wherein the pharmaceutical composition inhibits metastasis of the malignant cancer cells.

11. The method of claim 1, wherein the pharmaceutical composition further comprises a therapeutic agent.

12. The method of claim 11, wherein the therapeutic agent comprises an anti-PD1 antibody, a chemotherapeutic agent, or an immunotherapeutic agent.

13. The method of claim 12, wherein the ratio of the 1,3,5-triazine derivative to the therapeutic agent is 250:1.

14. The method of claim 1, wherein the therapeutically effective amount of the 1,3,5-triazine derivative is between 30 mg/kg to 50 mg/kg per day.

15. The method of claim 1, wherein the 1,3,5-triazine derivative is present in an amount of 30 mg to 50 mg per dosage unit.

16. The method of claim 1, wherein the pharmaceutical composition is administered orally.

17. The method of claim 1, wherein the pharmaceutical composition is administered via oral, intravenous, or intramuscular routes once a day.

18. The method of claim 1, wherein the pharmaceutical composition is in the form of powder, a tablet, a capsule, or an injectable solution.

19. A pharmaceutical composition comprising a 1,3,5-triazine derivative in an amount effective for treating a malignant cancer in a subject in need thereof, and a pharmaceutically acceptable carrier.

20. The pharmaceutical composition of claim 19, wherein the malignant cancer comprises a breast cancer, lung cancer, colorectal cancer, prostate cancer, pancreatic cancer, liver cancer, esophageal cancer, gastric cancer, ovarian cancer, cervical cancer, endometrial cancer, renal cancer, bladder cancer, thyroid cancer, melanoma, non-Hodgkin lymphoma, Hodgkin lymphoma, multiple myeloma, leukemia, glioblastoma, astrocytoma, medulloblastoma, meningioma, sarcoma, bone cancer, head and neck cancer, testicular cancer, oral cancer, anal cancer, mesothelioma, neuroblastoma, retinoblastoma, and cholangiocarcinoma.

21. The pharmaceutical composition of claim 19, wherein the 1,3,5-triazine derivative comprises the following structural formulas:

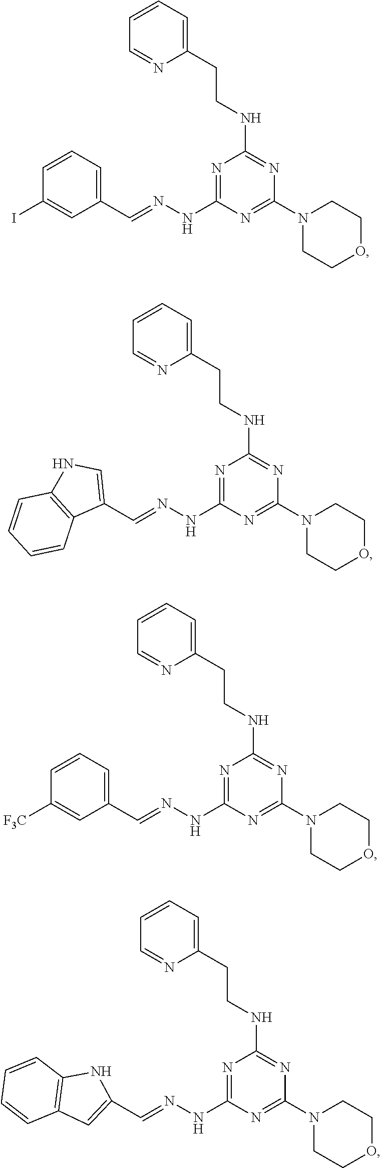 [SMILES: Ic1cccc(/C=N/Nc2nc(NCCc3ccccn3)nc(N3CCOCC3)n2)c1 ; C(=N\Nc1nc(NCCc2ccccn2)nc(N2CCOCC2)n1)/c1c[nH]c2ccccc12 ; FC(F)(F)c1cccc(/C=N/Nc2nc(NCCc3ccccn3)nc(N3CCOCC3)n2)c1 ; C(=N\Nc1nc(NCCc2ccccn2)nc(N2CCOCC2)n1)/c1cc2ccccc2[nH]1]

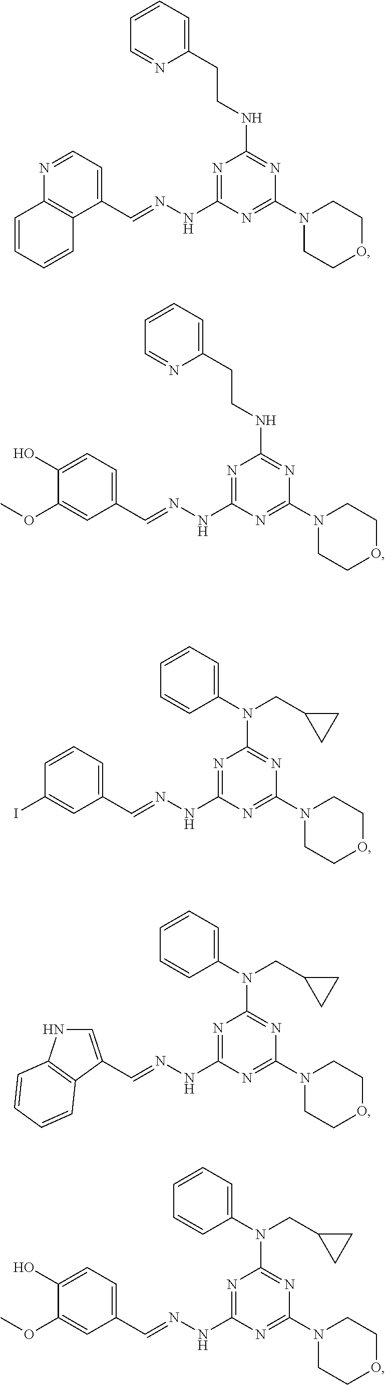 [SMILES: C(=N\Nc1nc(NCCc2ccccn2)nc(N2CCOCC2)n1)/c1ccnc2ccccc12 ; COc1cc(/C=N/Nc2nc(NCCc3ccccn3)nc(N3CCOCC3)n2)ccc1O ; Ic1cccc(/C=N/Nc2nc(N3CCOCC3)nc(N(CC3CC3)c3ccccc3)n2)c1 ; C(=N\Nc1nc(N2CCOCC2)nc(N(CC2CC2)c2ccccc2)n1)/c1c[nH]c2ccccc12 ; COc1cc(/C=N/Nc2nc(N3CCOCC3)nc(N(CC3CC3)c3ccccc3)n2)ccc1O]

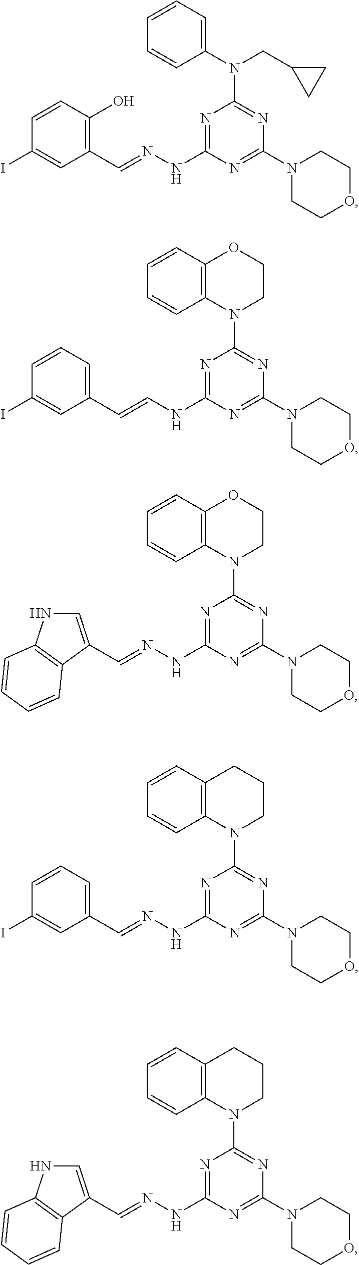 [SMILES: Oc1ccc(I)cc1/C=N/Nc1nc(N2CCOCC2)nc(N(CC2CC2)c2ccccc2)n1 ; Ic1cccc(/C=C/Nc2nc(N3CCOCC3)nc(N3CCOc4ccccc43)n2)c1 ; C(=N\Nc1nc(N2CCOCC2)nc(N2CCOc3ccccc32)n1)/c1c[nH]c2ccccc12 ; Ic1cccc(/C=N/Nc2nc(N3CCOCC3)nc(N3CCCc4ccccc43)n2)c1 ; C(=N\Nc1nc(N2CCOCC2)nc(N2CCCc3ccccc32)n1)/c1c[nH]c2ccccc12]

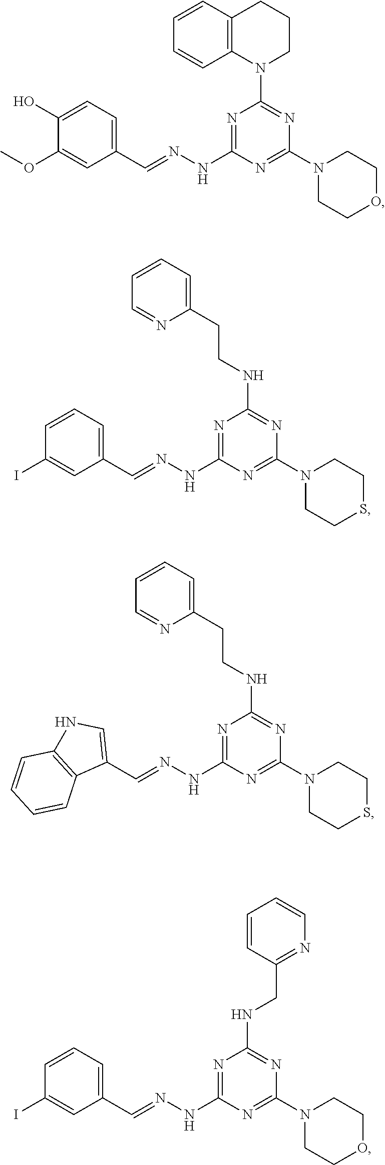 [SMILES: COc1cc(/C=N/Nc2nc(N3CCOCC3)nc(N3CCCc4ccccc43)n2)ccc1O ; Ic1cccc(/C=N/Nc2nc(NCCc3ccccn3)nc(N3CCSCC3)n2)c1 ; C(=N\Nc1nc(NCCc2ccccn2)nc(N2CCSCC2)n1)/c1c[nH]c2ccccc12 ; Ic1cccc(/C=N/Nc2nc(NCc3ccccn3)nc(N3CCOCC3)n2)c1]

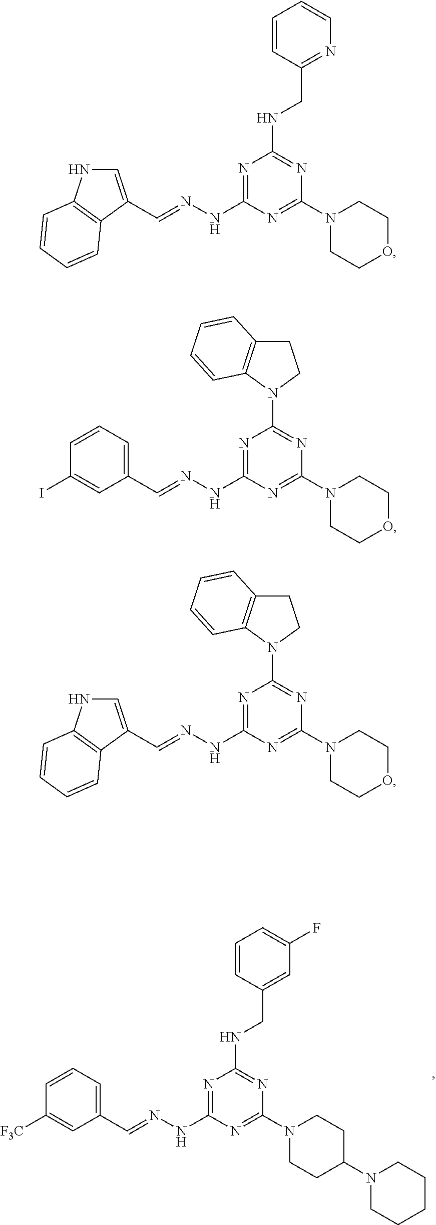 [SMILES: C(=N\Nc1nc(NCc2ccccn2)nc(N2CCOCC2)n1)/c1c[nH]c2ccccc12 ; Ic1cccc(/C=N/Nc2nc(N3CCOCC3)nc(N3CCc4ccccc43)n2)c1 ; C(=N\Nc1nc(N2CCOCC2)nc(N2CCc3ccccc32)n1)/c1c[nH]c2ccccc12 ; Fc1cccc(CNc2nc(N/N=C/c3cccc(C(F)(F)F)c3)nc(N3CCC(N4CCCCC4)CC3)n2)c1]

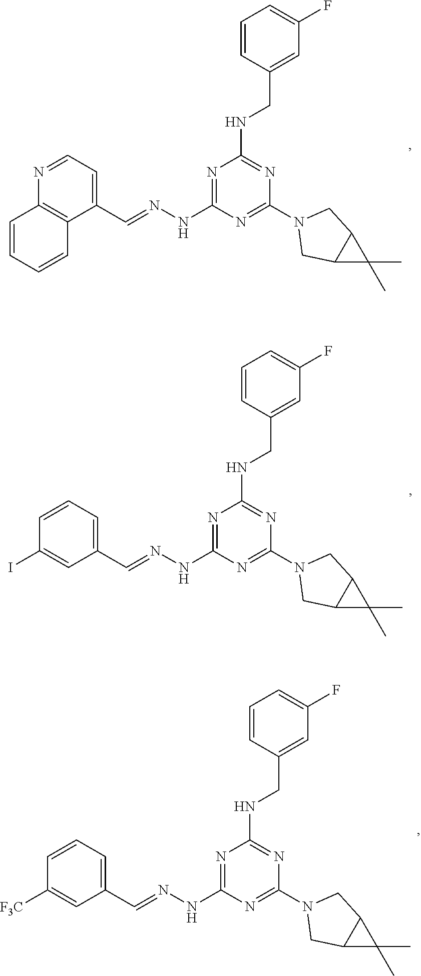 [SMILES: CC1(C)C2CN(c3nc(NCc4cccc(F)c4)nc(N/N=C/c4ccnc5ccccc45)n3)CC21 ; CC1(C)C2CN(c3nc(NCc4cccc(F)c4)nc(N/N=C/c4cccc(I)c4)n3)CC21 ; CC1(C)C2CN(c3nc(NCc4cccc(F)c4)nc(N/N=C/c4cccc(C(F)(F)F)c4)n3)CC21]

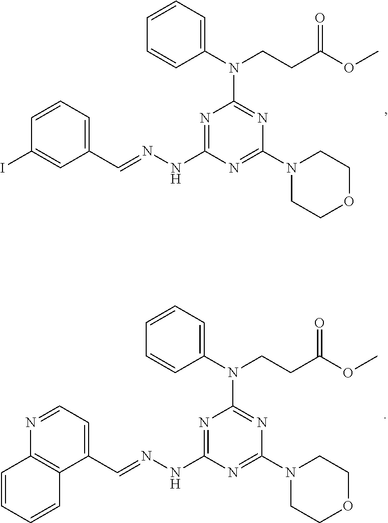 [SMILES: COC(=O)CCN(c1ccccc1)c1nc(N/N=C/c2cccc(I)c2)nc(N2CCOCC2)n1 ; COC(=O)CCN(c1ccccc1)c1nc(N/N=C/c2ccnc3ccccc23)nc(N2CCOCC2)n1]

22. The pharmaceutical composition of claim 19, wherein the pharmaceutically acceptable carrier is selected from the group consisting of diluents, excipients, and stabilizers.

23. The pharmaceutical composition of claim 19, wherein the pharmaceutical composition is administered via oral, intravenous, or intramuscular routes.
